(19) United States
(12) Patent Application Publication
(10) Pub. No.: US 2023/0193338 A1
(43) Pub. Date: Jun. 22, 2023

(54) GENETIC FACTOR TO INCREASE EXPRESSION OF RECOMBINANT PROTEINS

(71) Applicant: Impossible Foods Inc., Redwood City, CA (US)

(72) Inventors: Biswajoy Roy Chaudhuri, San Jose, CA (US); Svetlana Balatskaya, Fremont, CA (US); Martin Andrew Hoyt, San Francisco, CA (US)

(21) Appl. No.: 18/066,890

(22) Filed: Dec. 15, 2022

Related U.S. Application Data
(60) Provisional application No. 63/290,166, filed on Dec. 16, 2021.

(51) Int. Cl. C12P 21/00; C12N 15/81

(52) U.S. Cl. CPC C12P 21/00; C12N 15/815; C12N 2800/102

(57) ABSTRACT

Materials and methods that involve overexpression of a transcriptional activator such as retrograde regulation protein 1 (Rtg1) for increasing expression of one or more polypeptides.

### CLAIM OF PRIORITY

This application claims the benefit of U.S. Provisional Patent Application No. 63/290,166, filed on Dec. 16, 2021, which is incorporated by reference herein in its entirety.

### TECHNICAL FIELD

This disclosure generally relates to nucleic acid constructs and methods of using such to genetically engineer yeast cells (e.g., methylotrophic yeast cells).

### SEQUENCE LISTING

This application contains a Sequence Listing that has been submitted electronically as an XML file named “38767-0263001 SL ST26.XML.” The XML file, created on Dec. 8, 2022, is 64,642 bytes in size. The material in the XML file is hereby incorporated by reference in its entirety.

### BACKGROUND

Yeast cells such as Pichia pastoris are commonly used for expression of recombinant proteins. Constructs that can be used to efficiently express one or more proteins in a yeast cell (e.g., a methylotrophic yeast cell) are provided herein.

### SUMMARY

This disclosure describes the use of yeast strains that overexpress one or more transcriptional activators (e.g., Rtg1) to increase expression of transgenes that are expressed from a methanol utilization (mut) gene promoter, which significantly improves the recombinant production of one or more proteins. In addition, the effects of expression of combinations of transcriptional activators (e.g., Rtg1 and Mxr1) on mut gene promoter dependent gene expression was additive, thereby further increasing recombinant production of one or more proteins.

Accordingly, aspects of the present disclosure provide a yeast cell comprising: a first exogenous nucleic acid encoding a retrograde regulation protein (Rtg) operably linked to a first promoter element, and a second exogenous nucleic acid encoding a polypeptide operably linked to the first promoter element or a second promoter element. In some embodiments, the Rtg is Rtg1 or Rtg2 from Pichia pastoris or Saccharomyces cerevisiae.

In some embodiments, the polypeptide is selected from the group consisting of an antibody or fragment thereof, an enzyme, a regulatory protein, a peptide hormone, a blood clotting protein, a cytokine, a cytokine inhibitor, and a heme-binding protein. In some embodiments, the heme-binding protein is selected from the group consisting of a globin, a cytochrome, a cytochrome c oxidase, a ligninase, a catalase, and a peroxidase.

In some embodiments, the first exogenous nucleic acid, the second exogenous nucleic acid, or both the first exogenous nucleic acid and the second exogenous nucleic acid is stably integrated into the genome of the yeast cell. In some embodiments, the first exogenous nucleic acid, the second exogenous nucleic acid, or both the first exogenous nucleic acid and the second exogenous nucleic acid is extrachromosomally expressed from a replication-competent plasmid.

In some embodiments, the first promoter element is a constitutive promoter element. In some embodiments, the first promoter element, the second promoter element, or both the first promoter element and the second promoter element is an inducible promoter element.

In some embodiments, the inducible promoter element is a methanol-inducible promoter element. In some embodiments, the methanol-inducible promoter element is selected from the group consisting of an alcohol oxidase 1 (AOX1) promoter element from Pichia pastoris, an alcohol oxidase 2 (AOX2) promoter element from Pichia pastoris, a catalase 1 (CAT1) promoter from P. pastoris, a formate dehydrogenase (FMD) promoter from Hansenula polymorpha, an AOD1 promoter element from Candida boidinii, a FGH promoter element from Candida boidinii, a MOX promoter element from Hansenula polymorpha, a MODI promoter element from Pichia methanolica, a DHAS promoter element from Pichia pastoris, a FLD1 promoter element from Pichia pastoris, and a PEX8 promoter element from Pichia pastoris.

In some embodiments, the yeast cell further comprises a third exogenous nucleic acid encoding a transcriptional activator selected from methanol expression regulator 1 (Mxr1), methanol-induced transcription factor 1 (Mit1), and Trm1 operably linked to the first promoter element, the second promoter element, or a third promoter element. In some embodiments, the Mxr1, Mit1, or Trm1 transcriptional activator comprises a Mxr1, Mit1, or Trm1 element from Pichia pastoris. In some embodiments, the third promoter element is a constitutive promoter element or a methanol-inducible promoter element.

Aspects of the present disclosure provide a yeast cell comprising: a first exogenous nucleic acid encoding a first transcriptional activator selected from Rtg1, Rtg2, Mxr1, Mit1, and Trm1 operably linked to a first promoter element, a second exogenous nucleic acid encoding a second transcriptional activator selected from Rtg1, Rtg2, Mxr1, Mit1, and Trm1 operably linked to the first promoter element or a second promoter element, wherein the first transcriptional activator and the second transcriptional activator are different, and a third exogenous nucleic acid encoding a polypeptide operably linked to the first promoter element, the second promoter element, or a third promoter element.

In some embodiments, the yeast cell further comprises a fourth exogenous nucleic acid encoding one or more heme biosynthesis enzymes operably linked to the first promoter element, the second promoter element, the third promoter element, or a fourth promoter element. In some embodiments, the heme biosynthesis enzymes are selected from the group consisting of glutamate-1-semialdehyde (GSA) aminotransferase, 5-aminolevulinic acid (ALA) synthase, ALA dehydratase, porphobilinogen (PBG) deaminase, uroporphyrinogen (UPG) III synthase, UPG III decarboxylase, coproporphyrinogen (CPG) III oxidase, protoporphyrinogen (PPG) oxidase, and ferrochelatase. In some embodiments, the fourth promoter element is a constitutive promoter element or a methanol-inducible promoter element.

In some embodiments, the yeast cell is a methylotrophic yeast cell or a non-methylotrophic yeast cell. In some embodiments, the methylotrophic yeast cell is a Pichia cell. In some embodiments, the Pichia cell is a Pichia pastoris cell.

Aspects of the present disclosure provide a method for expressing a polypeptide, the method comprising: providing the yeast cell of any one of the preceding claims, and culturing the yeast cell under conditions suitable for expression of the first and the second exogenous nucleic acids or the first, second, and third exogenous nucleic acids.

In some embodiments, the culturing step comprises culturing the yeast cell in the presence of added iron or a pharmaceutically or metabolically acceptable salt thereof. In some embodiments, the culturing step comprises culturing the yeast cell in the absence or the presence of added methanol.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DETAILED DESCRIPTION

Nucleic acid constructs encoding transcriptional activators (e.g., Rtg1, Rtg2, Mxr1, Mit1, Trm1) are provided herein that allow for genetically engineering a yeast cell to increase the recombinant expression of a polypeptide. In some embodiments, the nucleic acid constructs provided herein allow for an increase in the recombinant expression of a polypeptide from an inducible promoter in the absence of the inducing molecule (e.g., methanol). Without being bound by any particular mechanism, the methods described herein create a positive feedback loop where the low-level native expression of one or more transcriptional activators turns on a mut promoter that is operably linked to one or more transcriptional activators. This leads to an increased expression of the one or more transcriptional activators as well as one or more target polypeptides that are operably linked to the same or different inducible promoters turned on by the one or more transcriptional activators. Alternatively, one or more transcriptional activators can be expressed from a constitutive promoter to turn on a mut promoter that is operably linked to one or more target polypeptides.

Accordingly, the present disclosure provides, in some aspects, nucleic acid constructs encoding one or more transcriptional activators (e.g., Rtg1, Rtg2, Mxr1, Mit1, Trm1) and methods of use thereof for producing a polypeptide.

### Transcriptional Activators

Methods and compositions described herein involve transcriptional activators (e.g., Rtg1, Rtg2, Mxr1, Mit1, Trm1) that increase expression of transgenes from mut gene promoters, thereby significantly improving the recombinant production of one or more proteins. Transcriptional activators and nucleic acids encoding transcriptional activators (e.g., exogenous nucleic acids encoding transcriptional activators) are known in the art and described herein. In some examples, the transcriptional activator can act on a mut gene promoter. In some examples, the transcriptional activator can function during carbon derepression. In some examples, the transcriptional activator can function during methanol induction. In some examples, the mut gene promoter has one or more binding sites for the transcriptional activator. In some examples, the transcriptional activator can be from a methylotrophic yeast. In some examples, the transcriptional activator can be from Pichia pastoris. In some examples, the transcriptional activator can be from Saccharomyces cerevisiae.

A representative P. pastoris Rtg1 nucleic acid sequence can be found, for example, in GenBank Accession No. XM_002489984.1 (see, e.g., SEQ ID NO: 1), while a representative P. pastoris Rtg1 polypeptide sequence can be found, for example, in GenBank Accession No. XP_002490029.1 (see, e.g., SEQ ID NO: 2).

A representative P. pastoris Rtg1 sequence can comprise one or more mutations. For example, a representative P. pastoris Rtg1 nucleic acid sequence comprises a mutation in GenBank Accession No. XM_002489984.1 (see, e.g., SEQ ID NO: 3). In another example, a representative P. pastoris Rtg1 polypeptide sequence comprises a mutation in GenBank Accession No. XP_002490029.1 (see, e.g., SEQ ID NO: 4).

A representative P. pastoris Rtg2 nucleic acid sequence can be found, for example, in GenBank Accession No. XM_002492633.1 (see, e.g., SEQ ID NO: 5), while a representative P. pastoris Rtg2 polypeptide sequence can be found, for example, in GenBank Accession No. XP_002492678.1 (see, e.g., SEQ ID NO: 6).

A representative P. pastoris methanol expression regulator 1 (Mxr1) nucleic acid sequence can be found, for example, in GenBank Accession No. DQ395124 (see, e.g., SEQ ID NO: 7), while a representative P. pastoris Mxr1 polypeptide sequence can be found, for example, in GenBank Accession No. ABD57365 (see, e.g., SEQ ID NO: 8).

A representative P. pastoris methanol-induced transcription factor 1 (Mit1) nucleic acid sequence can be found, for example, in GenBank Accession No. XM_002493021.1 (see, e.g., SEQ ID NO: 9), while a representative P. pastoris Mit1 polypeptide sequence can be found, for example, in GenBank Accession No. XP_002493066.1 (see, e.g., SEQ ID NO: 10). In some embodiments, the transcriptional activator is a Mit1 sequence from Pichia pastoris (see, e.g., GenBank Accession No. CAY70887).

A representative P. pastoris Trm1 nucleic acid sequence can be found, for example, in GenBank Accession No. XM_002493563.1 (see, e.g., SEQ ID NO: 11), while a representative P. pastoris Trm1 polypeptide sequence can be found, for example, in GenBank Accession No. XP_002493608.1 (see, e.g., SEQ ID NO: 12).

A representative S. cerevisiae Rtg1 nucleic acid sequence can be found, for example, in GenBank Accession No. XM_001183322.1 (see, e.g., SEQ ID NO: 13), while a representative S. cerevisiae Rtg1 polypeptide sequence can be found, for example, in GenBank Accession No. XP_014574.1 (see, e.g., SEQ ID NO: 14).

A representative S. cerevisiae Rtg2 nucleic acid sequence can be found, for example, in GenBank Accession No. XM_001181118.1 (see, e.g., SEQ ID NO: 15), while a representative S. cerevisiae Rtg2 polypeptide sequence can be found, for example, in GenBank Accession No. XP_011262.1 (see, e.g., SEQ ID NO: 16).

TABLE 1
Sequences of transcriptional activators.

| SEQ | | |
|---|---|---|
| ID | | |
| NO | Description | Sequence |
| 1 | P. pastoris | ATGGATAGTAATCAATGGCCCAAGGCGGAGCGTCCGTTCCAAGAAAATGAAATCTTGGAC |
| | Rtg1 (A427; | TTTTCCAGCTTGGATAATATACTCGACACTGATACTGAATTTGGAAGAAGTACCAGTAAA |
| | 1143) | CATGTACAACACACAGACCCCCCACTGCAACAGGACCAGTTGCTGACATACAATATAGAC |
| | | CAGGCGTCACAAAATACTCCCTCTCCTAACTTCTATCCTTCAAGCATTGATGTTAAGCAG |
| | | TCTCTTTCAAAGGCTTTACCCGCCTCGCATAATGTCAAGTCCGAATCTCCACAACAGGCC |
| | | GAGTACAACAGCAATGAGGATTCCAACAATCAATCCGAATCAAATATAAATACAGCGAAG |
| | | TCCCGGAGGAGCTCAGTGGTGACAACTCCAGGTGGGACTATTGTTGAGCGCAAGCGCAGA |
| | | GACAATATCAATGAACGTATACAGGACCTACTCACTGTTATTCCGGAGTCTTTTTTCCTA |
| | | GACCCCAAGGATAAAGCAAAAGCTACAGGTACCAAAGATGGAAAGCCTAATAAGGGGCAA |
| | | ATTTTAACAAAAGCAGTAGAGTATATTCATTGTCTTCAACAGGATATTGACGATAGAAAC |
| | | CGTCAAGAGGTCGCTTTGTCCTTGAAACTCAAAAACTTAGAGATTGCTCATAATGTACCG |
| | | GAAGAACGCAGAGAAGATTTAAAAAATACCTCTGCCGAAAAGGGCCTGGGTAGCATTGGT |
| | | GTTGGACCACTAGCAGATTGA |
| 2 | P. pastoris | MDSNQWPKAERPFQENEILDFSSLDNILDTDTEFGRSTSKHVQHTDPPLQQDQLLTYNID |
| | Rtg1 (A427; | QASQNTPSPNFYPSSIDVKQSLSKALPASHNVKSESPQQAEYNSNEDSNNQSESNINTAK |
| | 1143) | SRRSSVVTTPGGTIVERKRRDNINERIQDLLTVIPESFFLDPKDKAKATGTKDGKPNKGQ |
| | | ILTKAVEYIHCLQQDIDDRNRQEVALSLKLKNLEIAHNVPEERREDLKNTSAEKGLGSIG |
| | | VGPLAD |
| 3 | P. pastoris | ATGGATAGTAATCAATGGCCCAAGGCGGAGCGTCCGTTCCAAGAAAATGAAATCTTGGAC |
| | Rtg1 | TTTTCCAGCTTGGATAATATACTCGACACTGATACTGAATTTGGAAGAAGTACCAGTAAA |
| | (A427G; | CATGTACAACACACAGACCCCCCACTGCAACAGGACCAGTTGCTGACATACAATATAGAC |
| | 1143V) | CAGGCGTCACAAAATACTCCCTCTCCTAACTTCTATCCTTCAAGCATTGATGTTAAGCAG |
| | | TCTCTTTCAAAGGCTTTACCCGCCTCGCATAATGTCAAGTCCGAATCTCCACAACAGGCC |
| | | GAGTACAACAGCAATGAGGATTCCAACAATCAATCCGAATCAAATATAAATACAGCGAAG |
| | | TCCCGGAGGAGCTCAGTGGTGACAACTCCAGGTGGGACTATTGTTGAGCGCAAGCGCAGA |
| | | GACAATGTCAATGAACGTATACAGGACCTACTCACTGTTATTCCGGAGTCTTTTTTCCTA |
| | | GACCCCAAGGATAAAGCAAAAGCTACAGGTACCAAAGATGGAAAGCCTAATAAGGGGCAA |
| | | ATTTTAACAAAAGCAGTAGAGTATATTCATTGTCTTCAACAGGATATTGACGATAGAAAC |
| | | CGTCAAGAGGTCGCTTTGTCCTTGAAACTCAAAAACTTAGAGATTGCTCATAATGTACCG |
| | | GAAGAACGCAGAGAAGATTTAAAAAATACCTCTGCCGAAAAGGGCCTGGGTAGCATTGGT |
| | | GTTGGACCACTAGCAGATTGA |
| 4 | P. pastoris | MDSNQWPKAERPFQENEILDFSSLDNILDTDTEFGRSTSKHVQHTDPPLQQDQLLTYNID |
| | Rtg1 | QASQNTPSPNFYPSSIDVKQSLSKALPASHNVKSESPQQAEYNSNEDSNNQSESNINTAK |
| | (A427G; | SRRSSVVTTPGGTIVERKRRDNVNERIQDLLTVIPESFFLDPKDKAKATGTKDGKPNKGQ |
| | 1143V) | ILTKAVEYIHCLQQDIDDRNRQEVALSLKLKNLEIAHNVPEERREDLKNTSAEKGLGSIG |
| | | VGPLAD |
| 5 | P. pastoris | ATGTCCACAGTAGAGCTACAGGCAAATGAAGCTGAAATAGTAGCACGCTCATTGGTAGCC |
| | Rtg2 | ATCGTGGACATTGGTTCCAATGGAATCAGGTTTTCTGTGTCTTCCACCGCCTCCCATCAT |
| | | GCCAGAATTATGCCTTGTGTCTTCAAGGACAGATTGGGTATTTCACTGTTCGACGCCCAA |
| | | CTCGACAAGGGCTCTGCCAGTTCTATCAGCACACGTAAGCCGATACCCCAGGAAGCAATC |
| | | ACTGAGATCTGTTTGGCCATGAAACGATTCCAGTTGATTTGTGAGGATTTTGGAGTTTCA |
| | | AATGATAACGTGAAGATAGTTGCAACAGAAGCAACTAGGGAAGCCCCAAACTCTAAAGAA |
| | | TTCAGGGACGCAATTGCGAAGACCACAGGATGGGAAGTTGAATTGCTTTCAAAGGAAGAC |
| | | GAGGGCCGATGCGGTGCTTTCGGCGTTGCCTCCTCATTCCATAATATCTCTGGTATCTTC |
| | | ATGGATGTGGGGGGAGGATCTACTCAGCTGAGCTGGGTATCCACAGTCAATGGGGATGTC |
| | | AGACTTGCTGAATACCCTATATCTCTACCTTATGGGGCTGCTGCCCTTACTCAGCGATTA |
| | | TTATATGAAGATGAAAGAGAGGTTTATGAAGAGGTTCGTCAGGCTTATGAATTAGCGTTG |
| | | GAGAAGATAAAAATTCCTACAGAGCTCATCGAAGAAGCTGAAAAAAATGGCGGATTTAAT |
| | | TTGTATACTTGCGGCGGAGGATTCCGCGGTGTGGGACATCTTCTTCTTCATGAAGACCCA |
| | | AACTATCCAATTCAAACGATCATCAATGGTTACACAACTGGCTTCAAGAAAGTCGAATTG |
| | | TTGGCGAACTACCTTTTGTTGAAGAAAGAAGTTCCAAACTTCAGTGAAGGAAGCCCAAAG |
| | | ATTTTCAGAGTTTCAGAAAGACGAAAACAACAGCTCCCTGCTGTTGGACTACTGATGAGT |
| | | GCAGCATTCCAAGTGTTACCAAAAATTAGAACTGTCAGTTTCAGTGAAGGCGGTGTACGT |
| | | GAGGGTGTATTGTACAGTAGAATCTCACCATCTATAAGATCTGAAGATCCCCTTTTGACT |
| | | GCCACTCGTCCTTATGCTCCCCTTTTGTCTGAGCAATACAGGAAACTTCTTCTCGGTGCA |
| | | CTTCCAGAGGAAGTCCCCTCCGAGATCACCCAGACGATAGTACCTGCTCTTTGCAACATT |
| | | GCATTTGTCCACTGTTCATATCCTAAAGAGTTGCAACCAACAGCAGCGCTCCACATGGCT |
| | | ACCTCTGGTATTATCGCTGGAACTCATGGCCTTTCTCACAAAGTGCGTGCTCTAATAGGC |
| | | CTAGCATGTTGTGAACGTTGGGGGTTTGATCTTCCTGAATCAGAAGAAGTTTTTTACGGC |
| | | AAACTAGAAAAATTGGTTATTCAATCAGATCCAAATGACGGTGAAAGGTTACTATACTGG |
| | | ACAAAATATTGTGGAAAAATAATGTTTGTTATTTGCGGAGTACATCCCGGAGGAAACATA |
| | | CGTCCAGGTGTTATAGACTTCAACGTAATACCGCGGGCAGAAGCAAACAAGACCAACACG |
| | | GCTGTTCAAGTGGGAATGTCAGCCAATGATGTCAAATCGAGTTACACTGTTAAGAACAGA |
| | | ATTGCCAGTTTACAACGAAAAATCAAGAAACTGAACAAATCTTACAAAGGAAAAGACAGA |
| | | GTTGTGGTAGAGGTTGAGTATAGAATGTCATAG |
| 6 | P. pastoris | MSTVELQANEAEIVARSLVAIVDIGSNGIRFSVSSTASHHARIMPCVFKDRLGISLFDAQ |
| | Rtg2 | LDKGSASSISTRKPIPQEAITEICLAMKRFQLICEDFGVSNDNVKIVATEATREAPNSKE |
| | | FRDAIAKTTGWEVELLSKEDEGRCGAFGVASSFHNISGIFMDVGGGSTQLSWVSTVNGDV |
| | | RLAEYPISLPYGAAALTQRLLYEDEREVYEEVRQAYELALEKIKIPTELIEEAEKNGGFN |
| | | LYTCGGGFRGVGHLLLHEDPNYPIQTIINGYTTGFKKVELLANYLLLKKEVPNFSEGSPK |
| | | IFRVSERRKQQLPAVGLLMSAAFQVLPKIRTVSFSEGGVREGVLYSRISPSIRSEDPLLT |
| | | ATRPYAPLLSEQYRKLLLGALPEEVPSEITQTIVPALCNIAFVHCSYPKELQPTAALHMA |
| | | TSGIIAGTHGLSHKVRALIGLACCERWGFDLPESEEVFYGKLEKLVIQSDPNDGERLLYW |
| | | TKYCGKIMFVICGVHPGGNIRPGVIDFNVIPRAEANKTNTAVQVGMSANDVKSSYTVKNR |
| | | IASLQRKIKKLNKSYKGKDRVVVEVEYRMS |
| 7 | P. pastoris | ATGAGCAATCTACCCCCAACTTTTGGTTCCACTAGACAATCTCCAGAAGACCAATCACCT |
| | Mxr1 | CCCGTGCCCAAGGAGCTGTCATTCAATGGGACCACACCCTCAGGAAAGCTACGCTTATTT |
| | | GTCTGTCAGACATGTACTCGAGCATTTGCTCGTCAGGAACACTTGAAACGACACGAAAGG |
| | | TCTCACACCAAGGAGAAACCTTTCAGCTGCGGCATTTGTTCTCGTAAATTCAGCCGTCGA |
| | | GATCTGTTATTGAGACATGCCCAAAAACTGCACAGCAACTGCTCTGATGCGGCCATAACA |
| | | AGACTAAGGCGCAAGGCAACTCGTCGGTCTTCTAATGCCGCGGGTTCCATATCTGGTTCT |
| | | ACTCCGGTGACAACGCCAAATACTATGGGTACGCCCGAAGATGGCGAGAAACGAAAAGTT |
| | | CAGAAACTGGCCGGCCGCCGGGACTCAAATGAACAGAAACTGCAACTGCAACAACAACAT |
| | | CTACAGCAACAACCACAGTTGCAATACCAACAATCTCTTAAGCAGCATGAAAATCAAGTC |
| | | CAGCAGCCTGATCAAGATCCATTGATATCCCCGAGAATGCAATTATTCAATGATTCCAAC |
| | | CATCACGTAAACAATTTGTTTGATCTTGGACTAAGAAGAGCTTCCTTCTCCGCCGTTAGT |
| | | GGAAATAATTATGCCCATTATGTGAATAATTTTCAACAAGATGCCTCTTCTACCAATCCA |
| | | AATCAAGATTCAAATAATGCCGAATTTGAGAATATTGAATTTTCTACCCCACAAATGATG |
| | | CCCGTTGAAGATGCTGAAACTTGGATGAACAACATGGGTCCAATTCCGAACTTCTCTCTC |
| | | GATGTGAACAGGAACATTGGTGATAGCTTTACAGATATACAACACAAGAATTCAGAGCCT |
| | | ATTATATCCGAACCGCCCAAGGACACCGCTCCAAACGACAAGAAGTTGAATGGCTACTCT |
| | | TTTTACGAAGCCCCCATCAAGCCATTAGAATCCCTATTTTCTGTCAGGAATACAAAGAGA |
| | | AACAAGTATAAAACAAATGACGACTCTCCAGACACCGTGGATAATAACTCCGCACCGGCT |
| | | GCTAATACCATTCAAGAACTTGAGTCTTCTTTGAATGCATCCAAGAATTTTTGCTTGCCA |
| | | ACTGGTTATTCCTTCTATGGTAATTTGGACCAACAGACTTTCTCTAACACGTTATCATGC |
| | | ACTTCTTCTAATGCCACAATTTCGCCCATTCTACTCGATAACTCCATTAATAATAACTCC |
| | | ACTAGTGACGTGAGACCAGAATTTAGAACACAAAGTGTCACCTCTGAAATGAGTCAAGCC |
| | | CCTCCCCCTCCTCAAAAAAACAACTCGAAATATTCCACCGAAGTTCTTTTTACCAGCAAC |
| | | ATGCGGTCGTTTATTCACTACGCTCTTTCCAAGTATCCTTTTATTGGTGTGCCCACTCCA |
| | | ACTCTTCCGGAGAACGAAAGACTAAATGAATATGCTGATTCATTCACCAACCGTTTCTTA |
| | | AATCATTATCCTTTCATACATGTCACGATTCTCAAAGAATACTCCCTTTTCAAGGCAATT |
| | | TTAGATGAGAATGAGTCGACTAAGAACTGGGAAAATAATCAGTTTTACTTAGAGAACCAA |
| | | CGAATATCAATTGTTTGTCTTCCTCTTTTGGTGGCTACGATAGGTGCAGTACTATCAAAC |
| | | AACAAAAAGGATGCTTCGAATTTATACGAAGCTTCAAGGCGTTGTATTCATGTTTACTTA |
| | | GATTCCAGGAAAAAGATACCCACTTCCTTGTCCGCAAATAACAATGACTCTCCACTTTGG |
| | | CTAATTCAATCCCTGACGTTATCTGTTATGTATGGGTTATTTGCGGACAATGACATTAGT |
| | | TTGAATGTCGTGATCAGACAAGTTAACGCACTTAATTCTCTGGTCAAGACTTCGGGCCTG |
| | | AATAGGACCTCAATTATAGATCTTTTCAACATCAACAAACCTTTGGATAATGAACTCTGG |
| | | AATCAATTCGTGAAAATAGAGTCCACCGTAAGGACAATCCACACGATTTTTCAAATCAGT |
| | | TCCAACTTAAGCGCCTTGTACAATATTATTCCATCGTTGAAAATTGATGACCTAATGATT |
| | | ACTCTACCAGTTCCCACAACACTTTGGCAAGCTGATTCTTTTGTGAAATTCAAAAGTCTA |
| | | AGTTACGGAAATCAGATCCCTTTTCAATATACAAGAGTACTACAGAATTTGATTGATTAC |
| | | AATCAGCCATTGAGCGATGGAAAATTTTTGTATGAAAACCATGTAAGTGAGTTTGGACTC |
| | | ATATGCCTACAGAATGGTCTACACCAATACAGCTATTTCCAAAAATTGACTGCTGTCAAT |
| | | AACAGAGAAGATGCGCTATTCACAAAGGTTGTTAATTCACTTCACAGTTGGGATAGGATG |
| | | ATTTCGAATTCTGATTTGTTTCCAAAGAAGATATATCAGCAGAGTTGCTTGATTTTGGAC |
| | | TCAAAGTTGCTTAATAATTTCCTGATTGTCAAGAGCTCATTGAAAGTTTCGACCGGAGAC |
| | | GTTAGTTCTTTGAATAAGTTAAAAGAAAACGTGTGGCTTAAAAACTGGAATCAAGTGTGT |
| | | GCTATCTATTATAACAGCTTCATGAACATTCCTGCTCCCAGTATTCAAAAGAAGTACAAT |
| | | GACATAGAGTTTGTGGATGACATGATTAATTTGAGTCTAATCATCATCAAGATTATGAAA |
| | | CTCATTTTCTATAACAATGTCAAAGACAATTATGAGGATGAAAATGACTTCAAATTGCAA |
| | | GAGTTAAATTTAACATTTGACAATTTTGATGAGAAAATATCCTTGAATTTGACAATATTA |
| | | TTCGATATATTTTTGATGATCTACAAGATAATTACCAATTACGAAAAGTTTATGAAGATC |
| | | AAACACAAGTTTAATTACTACAATTCTAATTCGAATATAAGCTTCTTGCATCATTTCGAA |
| | | CTCTCCTCGGTTATCAATAACACCCAAATGAACCAGAATGATTATATGAAAACAGATATT |
| | | GATGAAAAGCTTGATCAGCTTTTCCACATCTATCAAACATTTTTCCGGCTGTATCTGGAT |
| | | TTAGAAAAGTTTATGAAGTTCAAATTCAACTATCATGACTTTGAGACAGAGTTTTCAAGT |
| | | CTCTCAATATCCAATATACTGAACACTCATGCTGCTTCTAACAATGACACAAATGCTGCT |
| | | GATGCTATGAATGCCAAGGATGAAAAAATATCTCCCACAACTTTGAATAGCGTATTACTT |
| | | GCTGATGAAGGAAATGAAAATTCCGGTCGTAATAACGATTCAGACCGCCTGTTCATGCTG |
| | | AACGAGCTAATTAATTTTGAAGTAGGTTTGAAATTTCTCAAGATAGGTGAGTCATTTTTT |
| | | GATTTCTTGTATGAGAATAACTACAAGTTCATCCACTTCAAAAACTTAAATGACGGAATG |
| | | TTCCACATCAGGATATACCTAGAAAACCGACTAGATGGTGGTGTCTAG |
| 8 | P. pastoris | MSNLPPTFGSTRQSPEDQSPPVPKELSFNGTTPSGKLRLFVCQTCTRAFARQEHLKRHER |
| | Mxr1 | SHTKEKPFSCGICSRKFSRRDLLLRHAQKLHSNCSDAAITRLRRKATRRSSNAAGSISGS |
| | | TPVTTPNTMGTPEDGEKRKVQKLAGRRDSNEQKLQLQQQHLQQQPQLQYQQSLKQHENQV |
| | | QQPDQDPLISPRMQLFNDSNHHVNNLFDLGLRRASFSAVSGNNYAHYVNNFQQDASSTNP |
| | | NQDSNNAEFENIEFSTPQMMPVEDAETWMNNMGPIPNFSLDVNRNIGDSFTDIQHKNSEP |
| | | IISEPPKDTAPNDKKLNGYSFYEAPIKPLESLFSVRNTKRNKYKTNDDSPDTVDNNSAPA |
| | | ANTIQELESSLNASKNFCLPTGYSFYGNLDQQTFSNTLSCTSSNATISPILLDNSINNNS |
| | | TSDVRPEFRTQSVTSEMSQAPPPPQKNNSKYSTEVLFTSNMRSFIHYALSKYPFIGVPTP |
| | | TLPENERLNEYADSFTNRFLNHYPFIHVTILKEYSLFKAILDENESTKNWENNQFYLENQ |
| | | RISIVCLPLLVATIGAVLSNNKKDASNLYEASRRCIHVYLDSRKKIPTSLSANNNDSPLW |
| | | LIQSLTLSVMYGLFADNDISLNVVIRQVNALNSLVKTSGLNRTSIIDLFNINKPLDNELW |
| | | NQFVKIESTVRTIHTIFQISSNLSALYNIIPSLKIDDLMITLPVPTTLWQADSFVKFKSL |
| | | SYGNQIPFQYTRVLQNLIDYNQPLSDGKFLYENHVSEFGLICLQNGLHQYSYFQKLTAVN |
| | | NREDALFTKVVNSLHSWDRMISNSDLFPKKIYQQSCLILDSKLLNNFLIVKSSLKVSTGD |
| | | VSSLNKLKENVWLKNWNQVCAIYYNSFMNIPAPSIQKKYNDIEFVDDMINLSLIIIKIMK |
| | | LIFYNNVKDNYEDENDFKLQELNLTFDNFDEKISLNLTILFDIFLMIYKIITNYEKFMKI |
| | | KHKFNYYNSNSNISFLHHFELSSVINNTQMNQNDYMKTDIDEKLDQLFHIYQTFFRLYLD |
| | | LEKFMKFKFNYHDFETEFSSLSISNILNTHAASNNDTNAADAMNAKDEKISPTTLNSVLL |
| | | ADEGNENSGRNNDSDRLFMLNELINFEVGLKFLKIGESFFDFLYENNYKFIHFKNLNDGM |
| | | FHIRIYLENRLDGGV |
| 9 | P. pastoris | ATGAGTACCGCAGCCCCAATCAAGGAAGAAAGCCAATTTGCCCATTTGACCCTAATGAAC |
| | Mit1 | AAGGATATACCTTCGAACGCAAAACAGGCAAAGTCGAAAGTTTCAGCGGCCCCTGCTAAG |
| | | ACGGGCTCCAAATCTGCTGGTGGATCTGGCAACAACAACGCTGCACCTGTGAAAAAAAGA |
| | | GTCCGCACGGGCTGTTTGACCTGCCGAAAGAAGCACAAGAAATGTGACGAGAACAGAAAC |
| | | CCAAAATGTGACTTTTGCACTTTGAAAGGCTTGGAATGTGTCTGGCCAGAGAACAATAAG |
| | | AAGAATATCTTCGTTAACAACTCCATGAAGGATTTCTTAGGCAAGAAAACGGTGGATGGA |
| | | GCTGATAGTCTCAATTTGGCCGTGAATCTGCAACAACAGCAGAGTTCAAACACAATTGCC |
| | | AATCAATCGCTTTCCTCAATTGGATTGGAAAGTTTTGGTTACGGCTCTGGTATCAAAAAC |
| | | GAGTTTAACTTCCAAGACTTGATAGGTTCAAACTCTGGCAGTTCAGATCCGACATTTTCA |
| | | GTAGACGCTGACGAGGCCCAAAAACTCGACATTTCCAACAAGAACAGTCGTAAGAGACAG |
| | | AAACTAGGTTTGCTGCCGGTCAGCAATGCAACTTCCCATTTGAACGGTTTCAATGGAATG |
| | | TCCAATGGAAAGTCACACTCTTTCTCTTCACCGTCTGGGACTAATGACGATGAACTAAGT |
| | | GGCTTGATGTTCAACTCACCAAGCTTCAACCCCCTCACAGTTAACGATTCTACCAACAAC |
| | | AGCAACCACAATATAGGTTTGTCTCCGATGTCATGCTTATTTTCTACAGTTCAAGAAGCA |
| | | TCTCAAAAAAAGCATGGAAATTCCAGTAGACACTTTTCATACCCATCTGGGCCGGAGGAC |
| | | CTTTGGTTCAATGAGTTCCAAAAACAGGCCCTCACAGCCAATGGAGAAAATGCTGTCCAA |
| | | CAGGGAGATGATGCTTCTAAGAACAACACAGCCATTCCTAAGGACCAGTCTTCGAACTCA |
| | | TCGATTTTCAGTTCACGTTCTAGTGCAGCTTCTAGCAACTCAGGAGACGATATTGGAAGG |
| | | ATGGGCCCATTCTCCAAAGGACCAGAGATTGAGTTCAACTACGATTCTTTTTTGGAATCG |
| | | TTGAAGGCAGAGTCACCCTCTTCTTCAAAGTACAATCTGCCGGAAACTTTGAAAGAGTAC |
| | | ATGACCCTTAGTTCGTCTCATCTGAATAGTCAACACTCCGACACTTTGGCAAATGGCACT |
| | | AACGGTAACTATTCTAGCACCGTTTCCAACAACTTGAGCTTAAGTTTGAACTCCTTCTCT |
| | | TTCTCTGACAAGTTCTCATTGAGTCCACCAACAATCACTGACGCCGAAAAGTTTTCATTG |
| | | ATGAGAAACTTCATTGACAACATCTCGCCATGGTTTGACACTTTTGACAATACCAAACAG |
| | | TTTGGAACAAAAATTCCAGTTCTGGCCAAAAAATGTTCTTCATTGTACTATGCCATTCTG |
| | | GCTATATCTTCTCGTCAAAGAGAAAGGATAAAGAAAGAGCACAATGAAAAAACATTGCAA |
| | | TGCTACCAATACTCACTACAACAGCTCATCCCTACTGTTCAAAGCTCAAATAATATTGAG |
| | | TACATTATCACATGTATTCTCCTGAGTGTGTTCCACATCATGTCTAGTGAACCTTCAACC |
| | | CAGAGGGACATCATTGTGTCATTGGCAAAATACATTCAAGCATGCAACATAAACGGATTT |
| | | ACATCTAATGACAAACTGGAAAAGAGTATTTTCTGGAACTATGTCAATTTGGATTTGGCT |
| | | ACTTGTGCAATCGGTGAAGAGTCAATGGTCATTCCTTTTAGCTACTGGGTTAAAGAGACA |
| | | ACTGACTACAAGACCATTCAAGATGTGAAGCCATTTTTCACCAAGAAGACTAGCACGACA |
| | | ACTGACGATGACTTGGACGATATGTATGCCATCTACATGCTGTACATTAGTGGTAGAATC |
| | | ATTAACCTGTTGAACTGCAGAGATGCGAAGCTCAATTTTGAGCCCAAGTGGGAGTTTTTG |
| | | TGGAATGAACTCAATGAATGGGAATTGAACAAACCCTTGACCTTTCAAAGTATTGTTCAG |
| | | TTCAAGGCCAATGACGAATCGCAGGGCGGATCAACTTTTCCAACTGTTCTATTCTCCAAC |
| | | TCTCGAAGCTGTTACAGTAACCAGCTGTATCATATGAGCTACATCATCTTAGTGCAGAAT |
| | | AAACCACGATTATACAAAATCCCCTTTACTACAGTTTCTGCTTCAATGTCATCTCCATCG |
| | | GACAACAAAGCTGGGATGTCTGCTTCCAGCACACCTGCTTCAGACCACCACGCTTCTGGT |
| | | GATCATTTGTCTCCAAGAAGTGTAGAGCCCTCTCTTTCGACAACGTTGAGCCCTCCGCCT |
| | | AATGCAAACGGTGCAGGTAACAAGTTCCGCTCTACGCTCTGGCATGCCAAGCAGATCTGT |
| | | GGGATTTCTATCAACAACAACCACAACAGCAATCTAGCAGCCAAAGTGAACTCATTGCAA |
| | | CCATTGTGGCACGCTGGAAAGCTAATTAGTTCCAAGTCTGAACATACACAGTTGCTGAAA |
| | | CTGTTGAACAACCTTGAGTGTGCAACAGGCTGGCCTATGAACTGGAAGGGCAAGGAGTTA |
| | | ATTGACTACTGGAATGTTGAAGAATAG |
| 10 | P. pastoris | MSTAAPIKEESQFAHLTLMNKDIPSNAKQAKSKVSAAPAKTGSKSAGGSGNNNAAPVKKR |
| | Mil1 | VRTGCLTCRKKHKKCDENRNPKCDFCTLKGLECVWPENNKKNIFVNNSMKDFLGKKTVDG |
| | | ADSLNLAVNLQQQQSSNTIANQSLSSIGLESFGYGSGIKNEFNFQDLIGSNSGSSDPTFS |
| | | VDADEAQKLDISNKNSRKRQKLGLLPVSNATSHLNGFNGMSNGKSHSFSSPSGTNDDELS |
| | | GLMFNSPSFNPLTVNDSTNNSNHNIGLSPMSCLFSTVQEASQKKHGNSSRHFSYPSGPED |
| | | LWFNEFQKQALTANGENAVQQGDDASKNNTAIPKDQSSNSSIFSSRSSAASSNSGDDIGR |
| | | MGPFSKGPEIEFNYDSFLESLKAESPSSSKYNLPETLKEYMTLSSSHLNSQHSDTLANGT |
| | | NGNYSSTVSNNLSLSLNSFSFSDKFSLSPPTITDAEKFSLMRNFIDNISPWFDTFDNTKQ |
| | | FGTKIPVLAKKCSSLYYAILAISSRQRERIKKEHNEKTLQCYQYSLQQLIPTVQSSNNIE |
| | | YIITCILLSVFHIMSSEPSTQRDIIVSLAKYIQACNINGFTSNDKLEKSIFWNYVNLDLA |
| | | TCAIGEESMVIPFSYWVKETTDYKTIQDVKPFFTKKTSTTTDDDLDDMYAIYMLYISGRI |
| | | INLLNCRDAKLNFEPKWEFLWNELNEWELNKPLTFQSIVQFKANDESQGGSTFPTVLFSN |
| | | SRSCYSNQLYHMSYIILVQNKPRLYKIPFTTVSASMSSPSDNKAGMSASSTPASDHHASG |
| | | DHLSPRSVEPSLSTTLSPPPNANGAGNKFRSTLWHAKQICGISINNNHNSNLAAKVNSLQ |
| | | PLWHAGKLISSKSEHTQLLKLLNNLECATGWPMNWKGKELIDYWNVEE |
| 11 | P. pastoris | ATGCCTCCTAAACATCGGCTGGAGCAGAGTATACAGCCCATGGCTTCTCAACAAATAGTA |
| | Trm1 | CCCGGTAATAAGGTTATTCTGCCGAATCCAAAAGTAGATGCAAAATCTACCCCAAACATT |
| | | TCAGTTCAGAAGAGAAGAAGAGTCACCAGAGCTTGTGATGAATGTCGGAAAAAGAAGGTC |
| | | AAATGTGATGGTCAACAACCATGCATTCATTGTACCGTTTATTCCTATGAGTGCACTTAC |
| | | AGCCAACCTTCCAGTAAGAAGAGACAGGGACAATCTCTGAGTCTGAGTGCTCCGTCAAAC |
| | | ATTAATGCAACAAGTTCCGTACAAAAATCTGTAAAACCTCCTGAAATCGATTTCCAAAGG |
| | | ATGAGAGACGCACTCAAATATTACGAAGATCTTTTAAACCAGTTGATATACCCCAACAGT |
| | | GCTCCAACTGTTCGAGTTAATCCGATTCGTCTAGCATCGATCTTAAAACAATTGAGAGCC |
| | | GATAAATCAAGTGATGAATTAATTTCAGTCAAGGCTCTTTCTGACAATTACATTGAGATG |
| | | CTTCACAAAACGATGCAACAACCTGTACAGCAGCCAGCTCCTCCTTCATTGGGGCAAGGA |
| | | GGGTCCTTCTCTAATCACAGTCCCAATCATAATAATGCTTCTATTGATGGTTCCATAGAA |
| | | TCTAATCTAGGGAGGGAAATACGTATCATATTACCTCCGAGAGATATTGCGCTGAAGCTT |
| | | ATCTACAAGACTTGGGACAACGCGTGTGTACTTTTCCGCTTTTATCACAGACCCGCATTT |
| | | ATTGAGGACCTGAATGAGTTATATGAAACAGATTTGGCAAACTACACCAATAAACAACAA |
| | | AGGTTTTTACCTCTTGTATATTCGGTGATGGCTTGTGGTGCTCTTTTTTGCAAGACTGAT |
| | | GGGATTAATCACGGCCAAAAGAGCTCCAAGCCCAAAGACTCTTCTGATGAAAGTCTCATA |
| | | GACGATGAGGGTTACAAGTATTTTATTGCCGCAAGAAAACTAATAGATATCACGGATACC |
| | | AGGGATACCTACGGAATTCAGACTATTGTTATGCTGATCATTTTTTTACAATGTTCGGCT |
| | | CGTCTTTCAACATGCTATTCTTATATTGGCATTGCTCTAAGAGCTGCATTGAGAGAAGGT |
| | | TTGCATCGTCAGTTGAACTATCCTTTCAATCCAATTGAGTTAGAAACAAGAAAGCGTCTT |
| | | TTTTGGACTATCTATAAAATGGACATCTATGTCAATACAATGCTGGGGCTTCCAAGAACC |
| | | ATTTCTGAAGAGGATTTCGACCAGGAAATGCCTATCGAACTTGATGATGAGAACATTAGT |
| | | GAAACCGGATATAGGTTCGATTTACAAGGTACAAAGTTATCCAGTTCAGGAATAGCCAAT |
| | | GCTCACACTAGATTGATATTCATAATGAAGAAAATTGTGAAAAAATTATATCCTGTCAAA |
| | | CTACAGAAACCAACCTCAAACAGTGGCGATACCCCACTTGAGAACAATGATTTATTGGCT |
| | | CATGAAATCGTTCATGAACTTGAGATGGATCTCCAAAATTGGGTCAATAGTCTACCTGCA |
| | | GAACTAAAACCGGGGATAGAACCACCGACCGAGTATTTTAAAGCTAACAGATTGCTTCAT |
| | | TTGGCATACCTGCATGTCAAGATTATTCTCTACAGGCCATTTATTCATTACATCTCAGAA |
| | | AAGGATAAGGTTGGAAATAGTTCTATCCCTCCGTCGCCCGAAGAGATCACTTCTATCGAG |
| | | AAAGCCAAGAATTGTGTCAATGTTGCCAGAATTGTTGTTAAACTAGCCGAAGACATGATT |
| | | AATAGGAAAATGTTAAGTGGTTCATATTGGTTTTCCATTTATACCATTTTTTTTTCCGTG |
| | | GCATGTCTGGTGTACTATGTTCATTTCGCTCCACCGAAGAAAGACAATGGAGAACTGGAT |
| | | CCCCAATACATGGAAATCAAGAAAGATACAGAGAGTGGAAGAGAGGTCTTAAATATCCTC |
| | | AAAGATAGTAGTATGGCGGCAAGAAGAACGTATAATATTCTCAACTCTTTGTTTGAGCAG |
| | | TTAAACAGAAGAACTGCAAAGGTCAACCTAGCAAAGGCACAGCAACCACCATCAGGGTTG |
| | | AATAACCCAGCTGCTACCCAGTATCAGAAACAGGGTGAACACAGGCAGTTACAACCAAGT |
| | | AACTATTCTGGAACTGTGAAATCTGTGGACCCAGAGAATATCGATTACTCTTCCTTTGGT |
| | | TCTCAGTTTGAAAACACTAACATCGAAGATGGTTCCTCAAATACAAAGATTGATCAGAAA |
| | | GTGAATGGGGTGAACTACATCGATGGTGTGTTTACAGGGATCAACCTAAATATGCCTAAT |
| | | CTCTCAGAAACTTCTAACACTCAAGGTATCGATAATCCAGCATTTCAAAGTATAAACAAT |
| | | TCTAATTTGAACAATAATTTTGTACAAACAAAGTACATTCCCGGCATGATGGACCAGCTA |
| | | GATATGAAAATTTTCGGAAGATTCCTTCCACCTTACATGCTGAACTCCAACAAGGTTGAA |
| | | CAGGGACAAAATGAAAGGAACCTATCAGGCCAACCATCCTCGTCGAATACTCCTGATGGA |
| | | TCACAACCTGTGACAGTTCTGGATGGATTATACCCGTTGCAGAATGATAATAATAATAAC |
| | | CACGACCCAGGAAATTCAAAGTCTGTTGTAAATAACAGTAACTCGGTAGAAAACTTACTA |
| | | CAGAACTTTACAATGGTGCCCTCGGGGTTGTCATCAACAGTGCAAAATCCTGAAGCGGCC |
| | | CAAAAATTCAATAATCATATGTCAAACATATCGAATATGAATGATCCAAGAAGAGCTAGC |
| | | GTAGCTACATCAGATGGATCCAATGACATGGATCATCATAGCCAAGGCCCGATAAACAAA |
| | | GATTTGAAACCGTTGAGCAACTACGAGTTTGACGATCTCTTCTTTAATGATTGGACCACT |
| | | GCGCCAGATACAATAAATTTTGACAGTTAA |
| 12 | P. pastoris | MPPKHRLEQSIQPMASQQIVPGNKVILPNPKVDAKSTPNISVQKRRRVTRACDECRKKKV |
| | Trm1 | KCDGQQPCIHCTVYSYECTYSQPSSKKRQGQSLSLSAPSNINATSSVQKSVKPPEIDFQR |
| | | MRDALKYYEDLLNQLIYPNSAPTVRVNPIRLASILKQLRADKSSDELISVKALSDNYIEM |
| | | LHKTMQQPVQQPAPPSLGQGGSFSNHSPNHNNASIDGSIESNLGREIRIILPPRDIALKL |
| | | IYKTWDNACVLFRFYHRPAFIEDLNELYETDLANYTNKQQRFLPLVYSVMACGALFCKTD |
| | | GINHGQKSSKPKDSSDESLIDDEGYKYFIAARKLIDITDTRDTYGIQTIVMLIIFLQCSA |
| | | RLSTCYSYIGIALRAALREGLHRQLNYPFNPIELETRKRLFWTIYKMDIYVNTMLGLPRT |
| | | ISEEDFDQEMPIELDDENISETGYRFDLQGTKLSSSGIANAHTRLIFIMKKIVKKLYPVK |
| | | LQKPTSNSGDTPLENNDLLAHEIVHELEMDLQNWVNSLPAELKPGIEPPTEYFKANRLLH |
| | | LAYLHVKIILYRPFIHYISEKDKVGNSSIPPSPEEITSIEKAKNCVNVARIVVKLAEDMI |
| | | NRKMLSGSYWFSIYTIFFSVACLVYYVHFAPPKKDNGELDPQYMEIKKDTESGREVLNIL |
| | | KDSSMAARRTYNILNSLFEQLNRRTAKVNLAKAQQPPSGLNNPAATQYQKQGEHRQLQPS |
| | | NYSGTVKSVDPENIDYSSFGSQFENTNIEDGSSNTKIDQKVNGVNYIDGVFTGINLNMPN |
| | | LSETSNTQGIDNPAFQSINNSNLNNNFVQTKYIPGMMDQLDMKIFGRFLPPYMLNSNKVE |
| | | QGQNERNLSGQPSSSNTPDGSQPVTVLDGLYPLQNDNNNNHDPGNSKSVVNNSNSVENLL |
| | | QNFTMVPSGLSSTVQNPEAAQKFNNHMSNISNMNDPRRASVATSDGSNDMDHHSQGPINK |
| | | DLKPLSNYEFDDLFFNDWTTAPDTINFDS |
| 13 | S. cerevisiae | ATGAGCAGCATTCCAGCTGGCACTGATCCTGGGTCCTGCGGTGCTAATTTCAAGAATGAC |
| | Rig1 | CGCAAGCGCAGAGATAAGATCAACGACCGTATTCAAGAACTATTGAGTATCATTCCCAAA |
| | | GACTTCTTTAGAGATTATTACGGCAATTCTGGTAGCAATGACACGTTAAGTGAATCCACT |
| | | CCCGGTGCGCTTGGGTTGTCCAGCAAGGCCAAAGGTACAGGGACCAAGGACGGAAAGCCC |
| | | AACAAGGGCCAAATTCTCACACAGGCGGTAGAGTACATATCACATCTACAAAATCAAGTG |
| | | GACACACAGAACAGAGAGGAGGTGGAACTGATGGTGAAGGCCACTCAGTTGGCCAAGCAG |
| | | ACAGGCACCATTGTCAACGATATAAACTTAGAGAACACCAGCGCTGAAGTCGCCCTGTCC |
| | | AGGATTGGCGTGGGACCGCTGGCCGCAACAAATGATGACTCAGTAAGACCGCCAGCAAAG |
| | | AGGTTGAGCTCCTTCGAGTACGGAGGGTATGGTGAGTACGGTAATGGTAGCTAA |
| 14 | S. cerevisiae | MSSIPAGTDPGSCGANFKNDRKRRDKINDRIQELLSIIPKDFFRDYYGNSGSNDTLSEST |
| | Rig1 | PGALGLSSKAKGTGTKDGKPNKGQILTQAVEYISHLQNQVDTQNREEVELMVKATQLAKQ |
| | | TGTIVNDINLENTSAEVALSRIGVGPLAATNDDSVRPPAKRLSSFEYGGYGEYGNGS |
| 15 | S. cerevisiae | ATGTCAACACTTAGCGATAGTGATACCGAGACTGAGGTCGTGTCGAGAAACTTGTGTGGA |
| | Rig2 | ATCGTCGACATAGGTTCTAATGGTATTCGTTTTAGTATATCTTCCAAGGCTGCACATCAT |
| | | GCAAGAATTATGCCTTGTGTTTTTAAAGATAGGGTTGGTCTTTCTCTATACGAAGTTCAA |
| | | TATAATACACATACGAACGCAAAATGCCCTATTCCCAGAGATATTATAAAAGAGGTTTGT |
| | | TCTGCCATGAAGAGATTCAAATTAATTTGCGATGATTTTGGTGTACCTGAAACTAGTGTC |
| | | AGAGTAATTGCAACAGAAGCCACGCGAGATGCTATTAACGCGGATGAATTTGTTAATGCT |
| | | GTTTACGGTAGCACTGGCTGGAAAGTAGAAATATTAGGCCAGGAAGATGAAACTAGGGTC |
| | | GGCATATATGGTGTTGTTTCCTCATTTAATACAGTAAGAGGTCTATATCTAGATGTGGCA |
| | | GGTGGTAGTACTCAGTTATCATGGGTAATAAGCTCGCACGGAGAAGTCAAGCAATCCAGC |
| | | AAACCTGTATCTTTGCCATATGGAGCTGGAACTCTTTTGAGAAGAATGAGAACAGATGAT |
| | | AATAGGGCACTTTTTTATGAGATTAAAGAAGCGTACAAAGATGCGATTGAAAAAATTGGT |
| | | ATACCTCAAGAAATGATTGATGACGCCAAGAAAGAAGGTGGATTTGACCTTTGGACCCGT |
| | | GGGGGTGGTTTAAGAGGTATGGGACATCTGCTTCTTTACCAGTCGGAAGGTTATCCCATC |
| | | CAAACAATAATTAACGGATATGCTTGCACTTATGAAGAATTCTCGTCTATGTCAGATTAT |
| | | CTATTCCTAAAACAAAAAATACCAGGTTCTTCAAAAGAGCATAAAATATTTAAGGTTTCT |
| | | GATAGAAGGGCTTTACAACTTCCTGCCGTTGGTTTGTTCATGAGTGCTGTTTTTGAAGCG |
| | | ATTCCCCAGATCAAAGCTGTACATTTTAGTGAGGGTGGTGTTCGAGAGGGTTCACTTTAT |
| | | TCTCTTCTTCCAAAAGAAATTCGTGCACAAGATCCATTGCTAATTGCGTCCCGTCCTTAT |
| | | GCTCCATTACTTACTGAAAAATATCTATATCTATTGAGAACATCAATCCCACAAGAAGAT |
| | | ATACCAGAAATAGTAAACGAAAGGATTGCTCCTGCTTTATGTAACTTAGCATTTGTTCAT |
| | | GCCTCTTATCCAAAGGAGTTACAACCAACAGCTGCATTACATGTTGCTACAAGAGGGATA |
| | | ATAGCCGGCTGTCATGGATTATCTCACAGAGCTAGAGCGCTGATAGGAATTGCTCTATGT |
| | | AGTAGATGGGGCGGCAACATTCCGGAATCTGAAGAAAAATACTCCCAAGAATTAGAACAA |
| | | GTAGTTCTACGCGAAGGTGATAAAGCTGAAGCATTGAGAATTGTATGGTGGACGAAGTAT |
| | | ATTGGTACGATTATGTATGTGATTTGCGGTGTTCATCCAGGTGGTAATATCAGAGATAAC |
| | | GTATTTGATTTCCATGTTTCTAAGCGTAGTGAGGTGGAGACCAGTTTAAAAGAATTAATC |
| | | ATTGATGATGCAAACACTACAAAGGTAAAAGAAGAATCCACGCGTAAAAATCGCGGGTAT |
| | | GAAGTGGTTGTGAGAATTAGTAAGGACGATCTTAAAACAAGTGCTTCCGTTCGTTCCAGA |
| | | ATTATCACGCTACAAAAGAAAGTACGCAAGCTATCTAGAGGAAGTGTAGAGAGGGTTAAA |
| | | ATTGGCGTGCAATTTTATGAAGAATAA |
| 16 | S. cerevisiae | MSTLSDSDTETEVVSRNLCGIVDIGSNGIRFSISSKAAHHARIMPCVFKDRVGLSLYEVQ |
| | Rtg2 | YNTHTNAKCPIPRDIIKEVCSAMKRFKLICDDFGVPETSVRVIATEATRDAINADEFVNA |
| | | VYGSTGWKVEILGQEDETRVGIYGVVSSFNTVRGLYLDVAGGSTQLSWVISSHGEVKQSS |
| | | KPVSLPYGAGTLLRRMRTDDNRALFYEIKEAYKDAIEKIGIPQEMIDDAKKEGGFDLWTR |
| | | GGGLRGMGHLLLYQSEGYPIQTIINGYACTYEEFSSMSDYLFLKQKIPGSSKEHKIFKVS |
| | | DRRALQLPAVGLFMSAVFEAIPQIKAVHFSEGGVREGSLYSLLPKEIRAQDPLLIASRPY |
| | | APLLTEKYLYLLRTSIPQEDIPEIVNERIAPALCNLAFVHASYPKELQPTAALHVATRGI |
| | | IAGCHGLSHRARALIGIALCSRWGGNIPESEEKYSQELEQVVLREGDKAEALRIVWWTKY |
| | | IGTIMYVICGVHPGGNIRDNVFDFHVSKRSEVETSLKELIIDDANTTKVKEESTRKNRGY |
| | | EVVVRISKDDLKTSASVRSRIITLQKKVRKLSRGSVERVKIGVQFYEE |

Suitable transcriptional activators also can be found in Hansenula polymorpha (the Adr1 sequence; see, e.g., GenBank Accession No. AEOI02000005, bases 858873 to 862352, for the nucleic acid sequence and GenBank Accession No. ESX01253 for the amino acid sequence; the Mpp1 sequence; see, e.g., GenBank Accession No. AY190521.1 for the nucleic acid sequence and GenBank Accession No. AAO72735.1 for the amino acid sequence) and Candida boidinii (the Trm1 sequence; see, e.g., GenBank Accession No. AB365355 for the nucleic acid sequence and GenBank Accession No. BAF99700 for the amino acid sequence; the Trm2 sequence; see, e.g., GenBank Accession No. AB548760 for the nucleic acid sequence and GenBank Accession No. BAJ07608 for the amino acid sequence; the HAP2 sequence; see, e.g., GenBank Accession No. AB909501.1 for the nucleic acid sequence and GenBank Accession No. BAQ21465.1 for the amino acid sequence; the HAP3 sequence; see, e.g., GenBank Accession No. AB909502.1 for the nucleic acid sequence and GenBank Accession No. BAQ21466.1 for the amino acid sequence; the HAPS sequence; see, e.g., GenBank Accession No. AB909503.1 for the nucleic acid sequence and GenBank Accession No. BAQ21467.1 for the amino acid sequence).

Combinations of two or more transcriptional activators can be used. In some examples, two, three, four, five, or more of Rtg1, Rtg2, Mxr1, Mit1, Trm1, Trm2, Adr1, Mpp1, HAP2, HAP3, HAP5, and any combination thereof are used in combination. In some examples, two, three, four, or five of Rtg1, Rtg2, Mxr1, Mit1, and Trm1 are used in combination. In some examples, Rtg1 and Rtg2 are used in combination. In some examples, Rtg1 and Mxr1 are used in combination. In some examples, Rtg1 and Mit1 are used in combination. In some examples, Rtg1 and Trm1 are used in combination. In some examples, Mit1 and Mxr1 are used in combination. In some examples, Mit1 and Trm1 are used in combination. In some examples, Mxr1 and Trm1 are used in combination. In some examples, Rtg1, Rtg2, and Mxr1 are used in combination. In some examples, Rtg1, Mxr1, and Mit1 are used in combination. In some examples, Rtg1, Rtg2, Mxr1, and Mit1 are used in combination.

Exogenous nucleic acids (e.g., nucleic acids encoding a polypeptide or transcriptional activator) may be placed under control of a promoter (e.g., those known in the art and described herein) that is inducible or constitutive. As used herein, “operably linked” means that a promoter or other expression element(s) are positioned relative to a nucleic acid coding sequence in such a way as to direct or regulate expression of the nucleic acid (e.g., in-frame).

### Products

Methods and compositions provided herein involve nucleic acid constructs for production of a product of interest (e.g., protein, DNA, RNA, or a small molecule of interest).

For example, a nucleic acid construct including a nucleotide sequence can be a nucleic acid construct encoding a protein. For example, a nucleic acid construct including a nucleotide sequence can be a nucleic acid construct encoding an RNA (e.g., an mRNA, a tRNA, a ribozyme, a siRNA, a miRNA, or a shRNA). For example, a nucleic acid construct including a nucleotide sequence can be a nucleic acid construct encoding a DNA. For example, in some embodiments, a nucleic acid construct including a nucleotide sequence can be a nucleic acid construct whose transcription results in or contributes to the production of a small molecule (e.g., heme, ethanol, a cofactor, a metabolite, a secondary metabolite, or a pharmaceutically active agent).

Accordingly, products produced using methods and compositions described herein can be widely used in many applications, such as for food, research, and medicine.

When the product is a polypeptide, the polypeptide can be a dehydrin, a phytase, a protease, a catalase, a lipase, a peroxidase, an amylase, a transglutaminase, an oxidoreductase, a transferase, a hydrolase, a lyase, an isomerase, or a ligase. In some embodiments, a polypeptide can be an antibody or fragment thereof (e.g., adalimumab, rituximab, trastuzumab, bevacizumab, infliximab, or ranibizumab), an enzyme (e.g., a therapeutic enzyme such as alpha-galactosidase A, alpha-L-iduronidase, N-acetylgalactosamine-4-sulfatase, dornase alfa, glucocerebrosidase, tissue plasminogen activator, rasburicase, an industrial enzyme (e.g., a catalase, a cellulase, a laccase, a glutaminase, or a glycosidase), a biocatalyst (e.g., an enzyme involved in biosynthesis or metabolism, a transaminase, a cytochrome P450, a kinase, a phosphorylase, or an isomerase)), a regulatory protein (e.g., a transcription factor (e.g., Mxr1), a peptide hormone (e.g., insulin, insulin-like growth factor 1, granulocyte colony-stimulating factor, follicle-stimulating hormone, or a growth hormone such as human growth hormone), a blood clotting protein (e.g., Factor VII), a cytokine (e.g., an interferon or erythropoietin), or a cytokine inhibitor (e.g., etanercept).

In some embodiments, a polypeptide can be a heme-binding protein (e.g., an exogenous or heterologous heme binding protein). In some embodiments, a heme-binding protein can be selected from the group consisting of a globin (PF00042 in the Pfam database), a cytochrome (e.g., a cytochrome P450, a cytochrome a, a cytochrome b, a cytochrome c), a cytochrome c oxidase, a ligninase, a catalase, and a peroxidase. In some embodiments, a globin can be selected from the group consisting of an androglobin, a chlorocruorin, a cytoglobin, an erythrocruorin, a flavohemoglobin, a globin E, a globin X, a globin Y, a hemoglobin (e.g., a beta hemoglobin, an alpha hemoglobin), a histoglobin, a leghemoglobin, a myoglobin, a neuroglobin, a non-symbiotic hemoglobin, a protoglobin, and a truncated hemoglobin (e.g., a HbN, a HbO, a Glb3, a cyanoglobin). In some embodiments, the heme-binding protein can be a myoglobin. In some embodiments, the heme-binding protein can be a hemoglobin. In some embodiments, the heme-binding protein can be a non-symbiotic hemoglobin. In some embodiments, the heme-binding protein can be a leghemoglobin. In some embodiments, the heme-binding protein can be soybean leghemoglobin (LegH). A reference amino acid sequence for LegH is provided in GenBank Accession No. NP_001235248.2 (see, e.g., SEQ ID NO: 20). LegH is a protein that binds to heme, which results in a characteristic absorption peak (Soret peak) at about 415 nm and a distinct red color. The LegH protein (also known as LGB2) is naturally found in root nodules of soybean. See, also, WO 2014/110539 and WO 2014/110532, each of which is incorporated by reference herein in its entirety. In some embodiments, a heme-binding protein can have an amino acid sequence that is at least 70% (e.g., at least 75%, 80%, 85%, 90%, 91%, 92%, 93%, 94%, 95%, 96%, 97%, 98%, or 99%) identical to the amino acid sequence set forth in any of SEQ ID NOs: 17-43. In some embodiments, a heme-binding protein is the amino acid sequence set forth in any of SEQ ID NOs: 17-43.

TABLE 2
Sequences of heme-binding proteins.

| SEQ | | |
|---|---|---|
| ID | | |
| NO | Description | Sequence |
| 17 | Non-symbiotic | MTTTLERGFTEEQEALVVKSWNVMKKNSGELGLKFFLKIFEIAPSAQKLFSFLRD |
| | hemoglobin [Vigna | STVPLEQNPKLKPHAVSVFVMTCDSAVQLRKAGKVTVRESNLKKLGATHFRTGVA |
| | radiata] | NEHFEVTKFALLETIKEAVPEMWSPAMKNAWGEAYDQLVDAIKYEMKPPSS |
| 18 | Hemoglobin-like | MIDQKEKELIKESWKRIEPNKNEIGLLFYANLFKEEPTVSVLFQNPISSQSRKLMQVLGIL |
| | flavoprotein | VQGIDNLEGLIPTLQDLGRRHKQYGVVDSHYPLVGDCLLKSIQEYLGQGFTEEAKAAWTKV |
| | [Methylacidiphilum | YGIAAQVMTAE |
| | infernorum] | |
| 19 | Hemoglobin-like | MLSEETIRVIKSTVPLLKEHGTEITARMYELLFSKYPKTKELFAGASEEQPKKLANAIIAY |
| | flavoprotein | ATYIDRLEELDNAISTIARSHVRRNVKPEHYPLVKECLLQAIEEVLNPGEEVLKAWEEAYD |
| | [Aquifex aeolicus] | FLAKTLITLEKKLYSQP |
| 20 | Leghemoglobin | MGAFTEKQEALVSSSFEAFKANIPQYSVVFYTSILEKAPAAKDLFSFLSNGVDPSNPKLTG |
| | [Glycine max] | HAEKLFGLVRDSAGQLKANGTVVADAALGSIHAQKAITDPQFVVVKEALLKTIKEAVGDKW |
| | | SDELSSAWEVAYDELAAAIKKAF |
| 21 | Non-symbiotic | MSAAEGAVVFSEEKEALVLKSWAIMKKDSANLGLRFFLKIFEIAPSARQMFPFLRDSDVPL |
| | hemoglobin | ETNPKLKTHAVSVFVMTCEAAAQLRKAGKITVRETTLKRLGGTHLKYGVADGHFEVTRFAL |
| | [Hordeum vulgare] | LETIKEALPADMWGPEMRNAWGEAYDQLVAAIKQEMKPAE |
| 22 | Heme peroxidase | MDGAVRLDWTGLDLTGHEIHDGVPIASRVQVMVSFPLFKDQHIIMSSKESPSRKSSTIGQS |
| | [Magnaporthe | TRNGSCQADTQKGQLPPVGEKPKPVKENPMKKLKEMSQRPLPTQHGDGTYPTEKKLTGIGE |
| | oryzae] | DLKHIRGYDVKTLLAMVKSKLKGEKLKDDKTMLMERVMQLVARLPTESKKRAELTDSLINE |
| | | LWESLDHPPLNYLGPEHSYRTPDGSYNHPFNPQLGAAGSRYARSVIPTVTPPGALPDPGLI |
| | | FDSIMGRTPNSYRKHPNNVSSILWYWATIIIHDIFWTDPRDINTNKSSSYLDLAPLYGNSQ |
| | | EMQDSIRTFKDGRMKPDCYADKRLAGMPPGVSVLLIMFNRFHNHVAENLALINEGGRFNKP |
| | | SDLLEGEAREAAWKKYDNDLFQVARLVTSGLYINITLVDYVRNIVNLNRVDTTWTLDPRQD |
| | | AGAHVGTADGAERGTGNAVSAEFNLCYRWHSCISEKDSKFVEAQFQNIFGKPASEVRPDEM |
| | | WKGFAKMEQNTPADPGQRTFGGFKRGPDGKFDDDDLVRCISEAVEDVAGAFGARNVPQAMK |
| | | VVETMGIIQGRKWNVAGLNEFRKHFHLKPYSTFEDINSDPGVAEALRRLYDHPDNVELYPG |
| | | LVAEEDKQPMVPGVGIAPTYTISRVVLSDAVCLVRGDRFYTTDFTPRNLTNWGYKEVDYDL |
| | | SVNHGCVFYKLFIRAFPNHFKQNSVYAHYPMVVPSENKRILEALGRADLFDFEAPKYIPPR |
| | | VNITSYGGAEYILETQEKYKVTWHEGLGFLMGEGGLKFMLSGDDPLHAQQRKCMAAQLYKD |
| | | GWTEAVKAFYAGMMEELLVSKSYFLGNNKHRHVD11RDVGNMVHVHFASQVFGLPLKTAKN |
| | | PTGVFTEQEMYGILAAIFTTIFFDLDPSKSFPLRTKTREVCQKLAKLVEANVKLINKIPWS |
| | | RGMFVGKPAKDEPLSIYGKTMIKGLKAHGLSDYDIAWSHVVPTSGAMVPNQAQVFAQAVDY |
| | | YLSPAGMHYIPEIHMVALQPSTPETDALLLGYAMEGIRLAGTFGSYREAAVDDVVKEDNGR |
| | | QVPVKAGDRVFVSFVDAARDPKHFPDPEVVNPRRPAKKYIHYGVGPHACLGRDASQIAITE |
| | | MFRCLFRRRNVRRVPGPQGELKKVPRPGGFYVYMREDWGGLFPFPVTMRVMWDDE |
| 23 | L-ascorbate | MKGSATLAFALVQFSAASQLVWPSKWDEVEDLLYMQGGFNKRGFADALRTCEFGSNVPGTQ |
| | peroxidase 5, | NTAEWLRTAFHDAITHDAKAGTGGLDASIYWESSRPENPGKAFNNTFGFFSGFHNPRATAS |
| | peroxisomal | DLTALGTVLAVGACNGPRIPFRAGRIDAYKAGPAGVPEPSTNLKDTFAAFTKAGFTKEEMT |
| | [Fusarium | AMVACGHAIGGVHSVDFPEIVGIKADPNNDTNVPFQKDVSSFHNGIVTEYLAGTSKNPLVA |
| | oxysporum] | SKNATFHSDKRIFDNDKATMKKLSTKAGFNSMCADILTRMIDTVPKSVQLTPVLEAYDVRP |
| | | YITELSLNNKNKIHFTGSVRVRITNNIRDNNDLAINLIYVGRDGKKVTVPTQQVTFQGGTS |
| | | FGAGEVFANFEFDTTMDAKNGITKFFIQEVKPSTKATVTHDNQKTGGYKVDDTVLYQLQQS |
| | | CAVLEKLPNAPLVVTAMVRDARAKDALTLRVAHKKPVKGSIVPRFQTAITNFKATGKKSSG |
| | | YTGFQAKTMFEEQSTYFDIVLGGSPASGVQFLTSQAMPSQCS |
| 24 | Cytochrome e | MASATRQFARAATRATRNGFAIAPRQVIRQQGRRYYSSEPAQKSSSAWIWLTGAAVAGGAG |
| | peroxidase | YYFYGNSASSATAKVFNPSKEDYQKVYNEIAARLEEKDDYDDGSYGPVLVRLAWHASGTYD |
| | [Fusarium | KETGTGGSNGATMRFAPESDHGANAGLAAARDFLQPVKEKFPWITYSDLWILAGVCAIQEM |
| | graminearum] | LGPAIPYRPGRSDRDVSGCTPDGRLPDASKRQDHLRGIFGRMGFNDQEIVALSGAHALGRC |
| | | HTDRSGYSGPWTFSPTVLTNDYFRLLVEEKWQWKKWNGPAQYEDKSTKSLMMLPSDIALIE |
| | | DKKFKPWVEKYAKDNDAFFKDFSNVVLRLFELGVPFAQGTENQRWTFKPTHQE |
| 25 | Group 1 truncated | MSLFAKLGGREAVEAAVDKFYNKIVADPTVSTYFSNTDMKVQRSKQFAFLAYALGGASEWK |
| | hemoglobin LI410 | GKDMRTAHKDLVPHLSDVHFQAVARHLSDTLTELGVPPEDITDAMAVVASTRTEVLNMPQQ |
| | [Chlamydomonas | |
| | eugametos] | |
| 26 | Hemoglobin | MNKPQTIYEKLGGENAMKAAVPLFYKKVLADERVKHFFKNTDMDHQTKQQTDFLTMLLGGP |
| | [Tetrahymena | NHYKGKNMTEAHKGMNLQNLHFDAIIENLAATLKELGVTDAVINEAAKVIEHTRKDMLGK |
| | pyriformis] | |
| 27 | Myoglobin | MSLFEQLGGQAAVQAVTAQFYANIQADATVATFFNGIDMPNQTNKTAAFLCAALGGPNAW |
| | [Paramecium | TGRNLKEVHANMGVSNAQFTTVIGHLRSALTGAGVAAALVEQTVAVAETVRGDVVTV |
| | caudatum] | |
| 28 | Hemoglobin | MPLTPEQIKIIKATVPVLQEYGTKITTAFYMNMSTVHPELNAVFNTANQVKGHQARALAG |
| | [Aspergillus niger] | ALFAYASHIDDLGALGPAVELICNKHASLYIQADEYKIVGKYLLEAMKEVLGDACTDDIL |
| | | DAWGAAYWALADIMINREAALYKQSQG |
| 29 | Hemoglobin [Zea | MALAEADDGAVVFGEEQEALVLKSWAVMKKDAANLGLRFFLKVFEIAPSAEQMFSFLRDS |
| | mays] | DVPLEKNPKLKTHAMSVFVMTCEAAAQLRKAGKVTVRETTLKRLGATHLRYGVADGHFEV |
| | | TGFALLETIKEALPADMWSLEMKKAWAEAYSQLVAAIKREMKPDA |
| 30 | Hemoglobin [Oryza | MALVEGNNGVSGGAVSFSEEQEALVLKSWAIMKKDSANIGLRFFLKIFEVAPSASQMFSFL |
| | saliva] | RNSDVPLEKNPKLKTHAMSVFVMTCEAAAQLRKAGKVTVRDTTLKRLGATHFKYGVGDAHF |
| | | EVTRFALLETIKEAVPVDMWSPAMKSAWSEAYNQLVAAIKQEMKPAE |
| 31 | Hemoglobin | MESEGKIVFTEEQEALVVKSWSVMKKNSAELGLKLFIKIFEIAPTTKKMFSFLRDSPIPA |
| | [Arabidopsis | EQNPKLKPHAMSVFVMCCESAVQLRKTGKVTVRETTLKRLGASHSKYGVVDEHFEVAKYA |
| | thaliana] | LLETIKEAVPEMWSPEMKVAWGQAYDHLVAAIKAEMNLSN |
| 32 | Leghemoglobin | MGFTDKQEALVNSSWESFKQNLSGNSILFYTIILEKAPAAKGLFSFLKDTAGVEDSPKLQA |
| | [Pisum sativum] | HAEQVFGLVRDSAAQLRTKGEVVLGNATLGAIHVQRGVTDPHFVVVKEALLQTIKKASGNN |
| | | WSEELNTAWEVAYDGLATAIKKAMT |
| 33 | Leghemoglobin | MVAFSDKQEALVNGAYEAFKANIPKYSVVFYTTILEKAPAAKNLFSFLANGVDATNPKLTG |
| | [Vigna unguiculata] | HAEKLFGLVRDSAAQLRASGGWADAALGAVHSQKAVNDAQFVWKEALVKTLKEAVGDKW |
| | | SDELGTAVELAYDELAAAIKKAY |
| 34 | Myoglobin [Bos | MGLSDGEWQLVLNAWGKVEADVAGHGQEVLIRLFTGHPETLEKFDKFKHLKTEAEMKASED |
| | taurus] | LKKHGNTVLTALGGILKKKGHHEAEVKHLAESHANKHKIPVKYLEFISDAIIHVLHAKHPS |
| | | DFGADAQAAMSKALELFRNDMAAQYKVLGFHG |
| 35 | Myoglobin [Sus | MGLSDGEWQLVLNVWGKVEADVAGHGQEVLIRLFKGHPETLEKFDKFKHLKSEDEMKASE |
| | scrofa] | DLKKHGNTVLTALGGILKKKGHHEAELTPLAQSHATKHKIPVKYLEFISEAIIQVLQSKH |
| | | PGDFGADAQGAMSKALELFRNDMAAKYKELGFQG |
| 36 | Myoglobin [Equus | MGLSDGEWQQVLNVWGKVEADIAGHGQEVLIRLFTGHPETLEKFDKFKHLKTEAEMKASED |
| | cabalius] | LKKHGTVVLTALGGILKKKGHHEAELKPLAQSHATKHKIPIKYLEFISDAIIHVLHSKHPG |
| | | DFGADAQGAMTKALELFRNDIAAKYKELGFQG |
| 37 | Hemoglobin | MSSFTEEQEALVLKSWDSMKKNAGEWGLKLFLKIFEIAPSAKKLFSFLKDSNVPLEQNAKL |
| | [Nicotiana | KPHAKSVFVMTCEAAVQLRKAGKVVVRDSTLKKLGAAHFKYGVADEHFEVTKFALLETIKE |
| | benthamiana] | AVPDMWSVDMKNAWGEAFDQLVNGIKTEMK |
| 38 | Hemoglobin | MGQSFNAPYEAIGEELLSQLVDTFYERVASHPLLKPIFPSDLTETARKQKQFLTQYLGGPP |
| | [Bacillus subtilis] | LYTEEHGHPMLRARHLPFPITNERADAWLSCMKDAMDHVGLEGEIREFLFGRLELTARHMV |
| | | NQTEAEDRSS |
| 39 | Globin | MTTSENFYDSVGGEETFSLIVHRFYEQVPNDDILGPMYPPDDFEGAEQRLKMFLSQYWGGP |
| | [Corynebacterium | KDYQEQRGHPRLRMRHVNYPIGVTAAERWLQLMSNALDGVDLTAEQREAIWEHMVRAADML |
| | glutamicum] | INSNPDPHA |
| 40 | Hemoglobin | MSTLYEKLGGTTAVDLAVDKFYERVLQDDRIKHFFADVDMAKQRAHQKAFLTYAFGGTDK |
| | [Synechocystis sp.] | YDGRYMREAHKELVENHGLNGEHFDAVAEDLLATLKEMGVPEDLIAEVAAVAGAPAHKRD |
| | | VLNQ |
| 41 | Globin | MDVALLEKSFEQISPRAIEFSASFYQNLFHHHPELKPLFAETSQTIQEKKLIFSLAAIIE |
| | [Synechococcus sp.] | NLRNPDILQPALKSLGARHAEVGTIKSHYPLVGQALIETFAEYLAADWTEQLATAWVEAY |
| | | DVIASTMIEGADNPAAYLEPELTFYEWLDLYGEESPKVRNAIATLTHFHYGEDPQDVQRD |
| | | SRG |
| 42 | Cyanoglobin | MSTLYDNIGGQPAIEQVVDELHKRIATDSLLAPVFAGTDMVKQRNHLVAFLAQIFEGPKQ |
| | [Nostoc commune] | YGGRPMDKTHAGLNLQQPHFDAIAKHLGERMAVRGVSAENTKAALDRVTNMKGAILNK |
| 43 | Globin [Bacillus | MREKIHSPYELLGGEHTISKLVDAFYTRVGQHPELAPIFPDNLTETARKQKQFLTQYLGGP |
| | megaterium] | SLYTEEHGHPMLRARHLPFEITPSRAKAWLTCMHEAMDEINLEGPERDELYHRLILTAQHM |
| | | INSPEQTDEKGFSH |

In some embodiments, a polypeptide can be a heme biosynthesis enzyme (e.g., an exogenous or heterologous heme biosynthesis enzyme). In some embodiments, a heme biosynthesis enzyme can be selected from the group consisting of glutamate-1-semialdehyde (GSA) aminotransferase, 5-aminolevulinic acid (ALA) synthase, ALA dehydratase, porphobilinogen (PBG) deaminase, uroporphyrinogen (UPG) III synthase, UPG III decarboxylase, coproporphyrinogen (CPG) III oxidase, protoporphyrinogen (PPG) oxidase, and ferrochelatase. See, also, U.S. Publication No. US20200340000A1, filed Apr. 24, 2020, which is incorporated herein by reference in its entirety.

Also provided are polypeptides that differ from a given sequence (e.g., those known in the art and described herein). Polypeptides can have at least 50% sequence identity (e.g., at least 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a given polypeptide sequence. In some embodiments, a polypeptide can have 100% sequence identity to a given polypeptide sequence.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the computer program ClustalW and default parameters, which allows alignments of nucleic acid or polypeptide sequences to be carried out across their entire length (global alignment). Chenna et al., 2003, Nucleic Acids Res., 31(13):3497-500. ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the default parameters can be used (i.e., word size: 2; window size: 4; scoring method: percentage; number of top diagonals: 4; and gap penalty: 5); for an alignment of multiple nucleic acid sequences, the following parameters can be used: gap opening penalty: 10.0; gap extension penalty: 5.0; and weight transitions: yes. For fast pairwise alignment of polypeptide sequences, the following parameters can be used: word size: 1; window size: 5; scoring method: percentage; number of top diagonals: 5; and gap penalty: 3. For multiple alignment of polypeptide sequences, the following parameters can be used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; and residue-specific gap penalties: on. ClustalW can be run, for example, at the Baylor College of Medicine Search Launcher website or at the European Bioinformatics Institute website on the World Wide Web.

### Promoters

Exogenous nucleic acids encoding the transcriptional activator (e.g., Rtg1, Rtg2, Mxr1, Mit1, Trm1) and/or the polypeptide can be operably linked to any promoter suitable for expression of the transcriptional activator and/or the polypeptide in yeast cells. As used herein, “operably linked” means that a promoter or other expression element(s) are positioned relative to a nucleic acid coding sequence in such a way as to direct or regulate expression of the nucleic acid (e.g., in-frame). The promoter can be a constitutive promoter or an inducible promoter (e.g., a methanol-inducible promoter).

Constitutive promoters and constitutive promoter elements are known in the art. For example, a commonly used constitutive promoter from P. pastoris is the promoter, or a portion thereof, from the transcriptional elongation factor EF-1α gene (TEF1), which is strongly transcribed in a constitutive manner. Other constitutive promoters, or promoter elements therefrom, however, can be used, including, without limitation, the glyceraldehyde-3-phosphate dehydrogenase (GAPDH or GAP) promoter from P. pastoris (see, e.g., GenBank Accession No. U62648.1), the promoter from the potential glycosylphosphatidylinositol (GPI)-anchored protein, GCW14p (PAS_chr1-4_0586), from P. pastoris (see, e.g., GenBank Accession No. XM_002490678), or the promoter from the 3-phosphoglycerate kinase gene (PGK1) from P. pastoris (see, e.g., GenBank Accession No. AY288296). Constitutive promoters and constitutive promoter elements from the host organism (e.g., a yeast cell such as a methylotrophic yeast cell or a non-methylotrophic yeast cell) can be used.

There are a number of inducible promoters that can be used when genetically engineering yeast. For example, a methanol-inducible promoter, or a promoter element therefrom, can be used. Methanol-inducible promoters are known in the art. For example, a commonly used methanol-inducible promoter from P. pastoris is the promoter, or a portion thereof, from the alcohol oxidase 1 (AOX1) gene, which is strongly transcribed in response to methanol. Other methanol-inducible promoters, or promoter elements therefrom, however, can be used, including, without limitation, the alcohol oxidase 2 (AOX2) promoter from P. pastoris (see, e.g., GenBank Accession No. X79871.1), the catalase 1 (CAT1) promoter from P. pastoris (see, e.g., Vogl et al., 2016, ACS Synth Biol 5:172-186), the formate dehydrogenase (FMD) promoter from Hansenula polymorpha, the alcohol oxidase (MOX) promoter from Hansenula polymorpha (see, e.g., GenBank Accession No. X02425), the alcohol oxidase (AOD1) promoter from Candida boidinii (see, e.g., GenBank Accession No. YSAAOD1A), the S-formylglutathione hydrolase (FGH) promoter from Candida boidinii, the MOD1 or MOD2 promoter from Pichia methanolica (see, e.g., Raymond et al., 1998, Yeast, 14:11-23; and Nakagawa et al., 1999, Yeast, 15:1223-30), the dihydroxyacetone synthase 1 or 2 (DHAS or DAS) promoter from P. pastoris (see, e.g., GenBank Accession No. FJ752551) or a promoter element therefrom, the formaldehyde dehydrogenase (FLD1) promoter from Pichia pastoris (see, e.g., GenBank Accession No. AF066054), the dihydroxyacetone kinase (DAK1) promoter from P. pastoris, or the peroxisomal matrix protein (PEX8) promoter from P. pastoris (see, e.g., Kranthi et al., 2010, Yeast, 27:705-11). In some embodiments, the methanol-inducible promoter is from a methylotrophic yeast. In some embodiments, the methanol-inducible promoter is a promoter of a gene in the methanol utilization pathway. In some embodiments, the methanol-inducible promoter is an alcohol oxidase promoter. All of these promoters are known to be induced by methanol.

Also within the scope of the present disclosure are nucleic acid constructs that include a promoter having a sequence that includes one or more mutations as compared to a reference promoter sequence. For example, expression from the Pichia pastoris promoter for the AOX1 gene (also referred to as pAOX1) is typically absent or very poor in the presence of non-inducing carbon sources (e.g., glucose or glycerol), and one or more mutations can be included in pAOX1 that allow significant expression from pAOX1 in the absence of methanol or in the absence of added methanol. In some examples, one or more mutations can be included in pAOX1 that allow an additional increase in expression from pAOX1 when methanol is present.

A reference pAOX1 sequence is provided in SEQ ID NO: 44. See, also, U.S. Publication No. US20200332267A1, filed Apr. 17, 2020, which is incorporated herein by reference in its entirety.

TABLE 3
pAOXI sequence.

| SEQ | | |
|---|---|---|
| ID | | |
| NO | Description | Sequence |
| 44 | Reference | AACATCCAAAGACGAAAGGT |
| | pAOXI | TGAATGAAACCTTTTTGCCA |
| | sequence | TCCGACATCCACAGGTCCAT |
| | | TCTCACACATAAGTGCCAAA |
| | | CGCAACAGGAGGGGATACAC |
| | | TAGCAGCAGACCGTTGCAAA |
| | | CGCAGGACCTCCACTCCTCT |
| | | TCTCCTCAACACCCACTTTT |
| | | GCCATCGAAAAACCAGCCCA |
| | | GTTATTGGGCTTGATTGGAG |
| | | CTCGCTCATTCCAATTCCTT |
| | | CTATTAGGCTACTAACACCA |
| | | TGACTTTATTAGCCTGTCTA |
| | | TCCTGGCCCCCCTGGCGAGG |
| | | TTCATGTTTGTTTATTTCCG |
| | | AATGCAACAAGCTCCGCATT |
| | | ACACCCGAACATCACTCCAG |
| | | ATGAGGGCTTTCTGAGTGTG |
| | | GGGTCAAATAGTTTCATGTT |
| | | CCCCAAATGGCCCAAAACTG |
| | | ACAGTTTAAACGCTGTCTTG |
| | | GAACCTAATATGACAAAAGC |
| | | GTGATCTCATCCAAGATGAA |
| | | CTAAGTTTGGTTCGTTGAAA |
| | | TGCTAACGGCCAGTTGGTCA |
| | | AAAAGAAACTTCCAAAAGTC |
| | | GGCATACCGTTTGTCTTGTT |
| | | TGGTATTGATTGACGAATGC |
| | | TCAAAAATAATCTCATTAAT |
| | | GCTTAGCGCAGTCTCTCTAT |
| | | CGCTTCTGAACCCCGGTGCA |
| | | CCTGTGCCGAAACGCAAATG |
| | | GGGAAACACCCGCTTTTTGG |
| | | ATGATTATGCATTGTCTCCA |
| | | CATTGTATGCTTCCAAGATT |
| | | CTGGTGGGAATACTGCTGAT |
| | | AGCCTAACGTTCATGATCAA |
| | | AATTTAACTGTTCTAACCCC |
| | | TACTTGACAGCAATATATAA |
| | | ACAGAAGGAAGCTGCCCTGT |
| | | CTTAAACCTTTTTTTTTATC |
| | | ATCATTATTAGCTTACTTTC |
| | | ATAATTGCGACTGGTTCCAA |
| | | TTGACAAGCTTTTGATTTTA |
| | | ACGACTTTTAACGACAACTT |
| | | GAGAAGATCAAAAAACAACT |
| | | AATTATTCGAAACG |

Also provided herein are nucleic acid constructs that include a promoter sequence having at least 70% (e.g., at least 75%, 80%, 85%, 90%, 95%, 97%, 98%, or 99%) sequence identity to a reference promoter sequence. For example, a promoter sequence can have at least 70% (e.g., at least 75%, 80%, 85%, 90%, 95%, 97%, 98%, or 99%) sequence identity to an alcohol oxidase promoter sequence (e.g., SEQ ID NO: 44). In some embodiments, a promoter sequence can have the sequence of SEQ ID NO: 44.

Also provided herein are nucleic acid constructs that include a promoter sequence having a sequence that includes one or more (e.g., 2, 3, 4, 5, 6, 7, 8, 9, 10, 11, 12, 13, 14, 15, 16, 17, 18, 19, 20, or more) mutations as compared to a reference promoter sequence.

### Nucleic Acids

Nucleic acid molecules used in the methods described herein are typically DNA molecules, but RNA molecules can be used under the appropriate circumstances. As used herein, “exogenous” refers to any nucleic acid sequence that is introduced into a cell from, for example, the same or a different organism or a nucleic acid generated synthetically (e.g., a codon-optimized nucleic acid sequence). For example, an exogenous nucleic acid can be a nucleic acid from one microorganism (e.g., one genus or species of yeast) that is introduced into a different genus or species of yeast; however, an exogenous nucleic acid also can be a nucleic acid from a yeast that is introduced recombinantly into a yeast as an additional copy despite the presence of a corresponding native nucleic acid sequence, or a nucleic acid from a yeast that is introduced recombinantly into a yeast containing one or more mutations, insertions, or deletions compared to the sequence native to the yeast. For example, P. pastoris contains an endogenous nucleic acid encoding an ALA synthase; an additional copy of the P. pastoris ALA synthase nucleic acid (e.g., introduced recombinantly into P. pastoris) is considered to be exogenous. Similarly, an “exogenous” protein is a protein encoded by an exogenous nucleic acid.

In some instances, an exogenous nucleic acid can be a heterologous nucleic acid. As used herein, a “heterologous” nucleic acid refers to any nucleic acid sequence that is not native to an organism (e.g., a heterologous nucleic acid can be a nucleic acid from one microorganism (e.g., one genus or species of yeast, whether or not it has been codon-optimized) that is introduced into a different genus or species of yeast)). Similarly, a “heterologous” protein is a protein encoded by a heterologous nucleic acid.

A nucleic acid molecule is considered to be exogenous to a host organism when any portion thereof (e.g., a promoter sequence or a sequence of an encoded protein) is exogenous to the host organism. A nucleic acid molecule is considered to be heterologous to a host organism when any portion thereof (e.g., a promoter sequence or a sequence of an encoded protein) is heterologous to the host organism.

Nucleic acid constructs are provided herein that allow for genetically engineering a yeast cell (e.g., a methylotrophic yeast cell). In some embodiments, nucleic acid constructs are provided herein that allow for genetically engineering a yeast cell (e.g., a methylotrophic yeast cell) to produce an RNA. Recombinantly produced RNAs can be used to modify a function of the cell, for example by RNA interference or as a guide for DNA editing. In some embodiments, nucleic acid constructs are provided herein that allow for genetically engineering a yeast cell (e.g., a methylotrophic yeast cell) to produce a product (e.g., a protein or small molecule), an exogenous product (e.g., an exogenous protein), a heterologous product (e.g., a heterologous protein), or a combination thereof. In some embodiments, nucleic acid constructs are provided herein that allow for genetically engineering a yeast cell (e.g., a methylotrophic yeast cell) to produce a product (e.g., a protein or small molecule) in the absence of methanol. In some embodiments, nucleic acid constructs are provided herein that allow for genetically engineering a yeast cell (e.g., a methylotrophic yeast cell) to produce a product (e.g., a protein or small molecule) in the presence of methanol. In addition, nucleic acid constructs are provided herein that allow for genetically engineering a yeast cell (e.g., a methylotrophic yeast cell) to increase the expression of a heme-binding protein and/or one or more heme biosynthesis enzymes.

A recombinant nucleic acid can include expression elements. Expression elements include nucleic acid sequences that direct and regulate expression of nucleic acid coding sequences. One example of an expression element is a promoter sequence. Expression elements also can include introns, enhancer sequences, insulators, silencers, operators, recognition sites, binding sites, cleavage sites, response elements, inducible elements, cis-regulatory elements, or trans-regulatory elements that modulate expression of a nucleic acid. Expression elements can be of bacterial, yeast, insect, mammalian, or viral origin, and vectors can contain a combination of elements from different origins.

It will be appreciated that a nucleic acid construct including a nucleotide sequence operably linked to any of the promoter elements as described herein can include a nucleotide sequence of interest. In some embodiments, transcription and/or translation of a nucleotide sequence can result in the production of a product (e.g., protein, DNA, RNA, or a small molecule) of interest. For example, in some embodiments, a nucleic acid construct including a nucleotide sequence can be a nucleic acid construct encoding a protein. For example, in some embodiments, a nucleic acid construct including a nucleotide sequence can be a nucleic acid construct encoding an RNA (e.g., an mRNA, a tRNA, a ribozyme, a siRNA, a miRNA, or a shRNA). For example, in some embodiments, a nucleic acid construct including a nucleotide sequence can be a nucleic acid construct encoding a DNA. For example, in some embodiments, a nucleic acid construct including a nucleotide sequence can be a nucleic acid construct whose transcription results in or contributes to the production of a small molecule (e.g., heme, ethanol, a cofactor, a metabolite, a secondary metabolite, or a pharmaceutically active agent).

In some embodiments, a nucleic acid construct (e.g., a first nucleic acid construct, a second nucleic acid construct, and so forth) including a nucleotide sequence can be a nucleic acid construct encoding a protein (e.g., a first protein, a second protein, and so forth).

Nucleic acid constructs described herein can be stably integrated into the genome of a yeast cell (e.g., methylotrophic yeast cell), or can be extrachromosomally expressed from a replication-competent plasmid. Methods of achieving both are well known and routinely used in the art.

In addition, it is noted that a first nucleic acid construct including a nucleotide sequence (e.g., encoding a first protein (e.g., a heme-binding protein)) operably linked to a promoter element (e.g., a promoter element as described herein) can be physically separate from a second nucleic acid construct including a nucleotide sequence (e.g., encoding a second protein (e.g., a transcription factor) operably linked to a promoter element (e.g., a promoter element as described herein) (that is, the first and second nucleic acid constructs can be completely separate molecules). Alternatively, a first nucleic acid construct including a nucleotide sequence (e.g., encoding a first protein) operably linked to a promoter element (e.g., a promoter element as described herein) and a second nucleic acid construct including a nucleotide sequence (e.g., encoding a second protein) operably linked to a promoter element (e.g., a promoter element as described herein) can be included in the same nucleic acid construct. In some embodiments, a first nucleic acid construct including a nucleotide sequence (e.g., encoding a first protein) operably linked to a promoter element can be contiguous with a second nucleic acid construct including a nucleotide sequence (e.g., encoding a second protein) operably linked to a promoter element. It would be appreciated by a skilled artisan that, if the second nucleic acid construct including a nucleotide sequence (e.g., encoding a second protein) is contiguous with the first nucleic acid construct including a nucleotide sequence (e.g., encoding a protein of interest), a single promoter, or promoter element therefrom, can be used to drive transcription of both or all of the nucleotide sequences (e.g., a nucleic acid encoding the first protein as well as a second protein). In some embodiments, a first nucleic acid construct can include two or more nucleotide sequences (e.g., encoding a first protein and a second protein (e.g., a heme-binding protein and a transcription factor, a heme-binding protein and a heme biosynthesis enzyme, two different transcription factors, or two different heme biosynthesis enzymes)) operably linked to one or more promoter elements (e.g., a promoter element as described herein), where the two or more nucleotide sequences can be contiguous or physically separate.

As used herein, nucleic acids can include DNA and RNA, and includes nucleic acids that contain one or more nucleotide analogs or backbone modifications. A nucleic acid can be single stranded or double stranded, which usually depends upon its intended use. Also provided are nucleic acids that differ from a given sequence. Nucleic acids can have at least 50% sequence identity (e.g., at least 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a given nucleic acid sequence. In some embodiments, a nucleic acid can have 100% sequence identity to a given nucleic acid sequence.

Also within the scope of the present disclosure is a construct or vector containing a nucleic acid construct as described herein (e.g., a nucleotide sequence that encodes a polypeptide operably linked to a promoter element as described herein). Constructs or vectors, including expression constructs or vectors, are commercially available or can be produced by recombinant DNA techniques routine in the art. A construct or vector containing a nucleic acid can have expression elements operably linked to such a nucleic acid, and further can include sequences such as those encoding a selectable marker (e.g., an antibiotic resistance gene). A construct or vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6× His tag, glutathione S-transferase (GST)).

### Mutations

Changes can be introduced into a nucleic acid molecule, thereby leading to changes in the amino acid sequence of the encoded polypeptide. For example, changes can be introduced into nucleic acid coding sequences using mutagenesis (e.g., site-directed mutagenesis, PCR-mediated mutagenesis, transposon mutagenesis, chemical mutagenesis, UV mutagenesis or radiation induced mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al., 1978, Atlas of Protein Sequence and Structure, 5(Suppl. 3):345-352, which provides frequency tables for amino acid substitutions), and a non-conservative substitution is one in which an amino acid residue is replaced with an amino acid residue that does not have a similar side chain. Nucleic acid and/or polypeptide sequences may be modified as described herein to improve one or more properties such as, without limitation, increased expression (e.g., transcription and/or translation), tighter regulation, deregulation, loss of catabolite repression, modified specificity, secretion, thermostability, solvent stability, oxidative stability, protease resistance, catalytic activity, and/or color.

In some embodiments, a mutation in a nucleic acid can be an insertion, a deletion or a substitution. In some embodiments, a mutation in a nucleic acid can be a substitution (e.g., a guanosine to cytosine mutation). In some embodiments, a mutation in a nucleic acid can be in a non-coding sequence. In some embodiments, a substitution in a coding sequence (e.g., encoding a protein) can be a silent mutation (e.g., the same amino acid is encoded). In some embodiments, a substitution in a coding sequence can be a nonsynonymous mutation (e.g., a missense mutation or a nonsense mutation). In some embodiments, a substitution in a coding sequence can be a missense mutation (e.g., a different amino acid is encoded). In some embodiments, a substitution in a coding sequence can be nonsense mutation (e.g., a premature stop codon is encoded). It will be understood that mutations can be used to alter an endogenous nucleic acid, using, for example, CRISPR, TALEN, and/or Zinc-finger nucleases.

In some embodiments, a mutation in a protein sequence can be an insertion, a deletion, or a substitution. It will be understood that a mutation in a nucleic acid that encodes a protein can cause a mutation in a protein sequence. In some embodiments, a mutation in a protein sequence is a substitution (e.g., a cysteine to serine mutation, or a cysteine to alanine mutation).

As used herein, a “corresponding” nucleic acid position (or substitution) in a nucleic acid sequence different from a reference nucleic acid sequence (e.g., in a truncated, extended, or mutated nucleic acid sequence) can be identified by performing a sequence alignment between the nucleic acid sequences of interest. It will be understood that in some cases, a gap can exist in a nucleic acid alignment. Similarly, a “corresponding” amino acid position (or substitution) in a protein sequence different from a reference protein sequence (e.g., in the myoglobin protein sequence of a different organism compared to a reference myoglobin protein sequence, such as SEQ ID NO: 34) can be identified by performing a sequence alignment between the protein sequences of interest. It will be understood that in some cases, a gap can exist in a protein alignment. As used herein, a nucleotide or amino acid position “relative to” a reference sequence can be the corresponding nucleotide or amino acid position in a reference sequence.

In some embodiments, a reference sequence can be from the same taxonomic rank as a comparator sequence. In some embodiments, a reference sequence can be from the same domain as a comparator sequence. For example, in some embodiments, both a reference sequence and a comparator sequence can be from domain Eukarya. In some embodiments, a reference sequence can be from the same kingdom as a comparator sequence. For example, in some embodiments, both a reference sequence and a comparator sequence can be from the kingdom Fungi. In some embodiments, a reference sequence can be from the same phylum as a comparator sequence. For example, in some embodiments, both a reference sequence and a comparator sequence can be from phylum Ascomycota. In some embodiments, a reference sequence can be from the same class as a comparator sequence. For example, in some embodiments, both a reference sequence and a comparator sequence can be from the class Saccharomycetes. In some embodiments, a reference sequence can be from the same order as a comparator sequence. For example, in some embodiments, both a reference sequence and a comparator sequence can be from the order Saccharomycetales. In some embodiments, a reference sequence can be from the same family as a comparator sequence. For example, in some embodiments, both a reference sequence and comparator sequence can be from the family Saccharomycetaceae. In some embodiments, a reference sequence can be from the same genus as a comparator sequence. For example, in some embodiments, both a reference sequence and a comparator sequence can be from the genus Pichia. In some embodiments, a reference sequence can be from the same species as a comparator sequence.

In some embodiments, a reference sequence and a comparator sequence can both be from yeast (e.g., methylotrophic yeast). In some embodiments, a reference sequence and a comparator sequence can have at least 50% (e.g., at least 55%, 60%, 65%, 70%, 75%, 80%, 85%, 90%, or 99%) sequence identity.

### Yeast Cells

Also provided herein is a yeast cell including any of the nucleic acid constructs described herein. A yeast cell can be any yeast cell suitable for producing one or more polypeptides. Non-limiting examples of yeast cells include Pichia (e.g., Pichia methanolica, Pichia pastoris) cells, Candida (e.g., Candida boidinii) cells, Hansenula (e.g., Hansenula polymorpha) cells, Torulopsis cells, and Saccharomyces (e.g., Saccharomyces cerevisiae) cells. In some embodiments, a yeast cell can be a methylotrophic yeast cell. Non-limiting examples of methylotrophic yeast cells include Pichia cells, Candida cells, Hansenula cells, and Torulopsis cells. In some embodiments, a yeast cell can be a Pichia cell or a Saccharomyces cell. The methylotrophic yeast cell can be a Pichia cell, a Candida cell, a Hansenula cell, or a Torulopsis cell. The methylotrophic yeast cell can be a Pichia methanolica cell, a Pichia pastoris cell, a Candida boidinii cell, or a Hansenula polymorpha cell. The methylotrophic yeast cell can be a Pichia pastoris cell. In some embodiments, a yeast cell can be a non-methylotrophic yeast cell. The non-methylotrophic yeast cell can be a Saccharomyces (e.g., Saccharomyces cerevisiae) cell, a Yarrowia lipolytica cell, a Kluyveromyces lactis cell, a Kluyveromyces marxianus cell, an Arxula adeninivorans cell, a Saccharomyces occidentalis cell, a Schizosaccharomyces pombe cell, a Pichia stipites cell, a Zygosaccharomyces bailii cell, or a Zygosaccharomyces rouxii cell.

Genetically engineering a yeast cell typically includes introducing a recombinant nucleic acid construct into the yeast cell. Accordingly, in some embodiments, a yeast cell described herein comprises a nucleic acid construct (e.g., a first nucleic acid construct, a second nucleic acid construct, and so forth) including a nucleotide sequence operably linked to a promoter element as described herein. As used herein, “operably linked” means that a promoter or other expression element(s) are positioned relative to a coding sequence in such a way as to direct or regulate expression of the coding sequence (e.g., in-frame). A nucleic acid construct including a nucleotide sequence can include any nucleotide sequence suitable for producing a polypeptide of interest.

### Methods for Producing Products

Also provided herein are methods of producing a product (e.g., a protein or small molecule) using any of the nucleic acid constructs and/or cells described herein. Such methods include culturing yeast cells comprising any one or more of the nucleic acids described herein. Methods of introducing nucleic acids into yeast cells are known in the art, and include, without limitation, transduction, electroporation, biolistic particle delivery, and chemical transformation.

Methods of culturing yeast cells are known in the art. See, e.g., Pichia Protocols, Methods In Molecular Biology, 389, Cregg, Ed., 2007, 2nd Ed., Humana Press, Inc. Under some circumstances, it may be desirable to introduce or add methanol to the culture media, although methanol is not required to obtain efficient expression at high levels of one or more polypeptides of interest. Under some circumstances (e.g., when one or more nucleic acids encoding enzyme(s) involved in an iron-co-factor biosynthesis are expressed), it may be desirable to supplement the culture media with iron or a pharmaceutically or metabolically acceptable (or GRAS) salt thereof.

Methods provided herein also can include purifying an expressed protein. As used herein, an “enriched” protein is a protein that accounts for at least 5% (e.g., at least 6%, 7%, 8%, 9%, 10%, 15%, 20%, 25%, 30%, 35%, 40%, 45%, 50%, 55%, 60%, or more) by dry weight, of the mass of the production cell, or at least 10% (e.g., at least 15%, 20%, 25%, 30%, 35%, 40%, 45%, 50%, 55%, 60%, 65%, 70%, 75%, 80%, 90%, 95%, or 99%) by dry weight, the mass of the production cell lysate (e.g., excluding cell wall or membrane material). As used herein, a “purified” protein is a protein that has been separated from cellular components that naturally accompany it. Typically, the protein is considered “purified” when it is at least 70% (e.g., at least 75%, 80%, 85%, 90%, 95%, or 99%) by dry weight, free from other proteins and naturally occurring molecules with which it is naturally associated.

Methods are described herein that can be used to generate a strain that lacks sequences for selection (i.e., that lacks a selectable marker). These methods include using a circular plasmid DNA vector and a linear DNA sequence; the circular plasmid DNA vector contains a selection marker and an origin of DNA replication (also known as an autonomously replicating sequence (ARS)), and the linear DNA sequence contains sequences for integration into the yeast cell genome by homologous recombination. A linear DNA molecule additionally can include nucleic acid sequences encoding one or more proteins of interest such as, without limitation, a heme-binding protein, a dehydrin, a phytase, a protease a catalase, a lipase, a peroxidase, an amylase, a transglutaminase, an oxidoreductase, a transferase, a hydrolase, a lyase, an isomerase, a ligase, one or more enzymes involved in the pathway for production of small molecules, such as heme, ethanol, lactic acid, butanol, adipic acid or succinic acid, or an antibody against any such proteins.

Yeast cells (e.g., methylotrophic yeast cells (e.g., Pichia)) can be transformed with both the circular plasmid DNA vector and the linear DNA sequence, and the transformants selected by the presence of the selectable marker on the circular plasmid. Transformants then can be screened for integration of the linear DNA molecule into the genome using, for example, PCR. Once transformants with the correct integration of the marker-free linear DNA molecule are identified, the cells can be grown in the absence of selection for the circular plasmid. Because the marker-bearing plasmid is not stably maintained in the absence of selection, the plasmid is lost, often very quickly, after selection is relaxed. The resulting strain carries the integrated linear DNA in the absence of heterologous sequences for selection. Therefore, this approach can be used to construct strains (e.g., Pichia strains) that lack a selectable marker (e.g., a heterologous selection marker) with little to no impact on recombinant product (e.g., protein) yield. Other methods such as Cre-Lox recombination, FLT-FRT recombination, or CRISPR-Cas9 can also be used to construct marker-free strains.

Methods provided herein allow for an increase in the titer of a product (e.g., a protein or small molecule). In some embodiments, the titer of a product (e.g., a protein or small molecule) can be increased by at least 5% (e.g., at least 6%, 7%, 8%, 9%, 10%, 15%, 20%, 30%, 40%, 50%, 60%, 70%, 80%, 90%, 100%, 150%, 200%, 250%, 300%, 350%, 400%, 500%, 600%, 700% , 800%, 900%, 1000%, or more) compared to a corresponding method lacking a nucleic acid construct as described herein.

Generally, a “titer” is the measurement of the amount of a substance in solution. As used herein, the “titer” of a product (e.g., a protein or small molecule) refers to the overall amount of the product. When the product is a heme-binding protein, the titer refers to the overall amount of the polypeptide whether or not it is bound to heme, unless otherwise specified. The titer of a product (e.g., a protein or small molecule) can be measured by any suitable method, such as high performance liquid chromatography (HPLC), high-performance liquid chromatography-mass spectrometry (HPLC MS), enzyme-linked immunosorbent assay (ELISA), or ultraviolet and/or visible light (UV-Vis) spectroscopy.

As used herein, a “corresponding method” is a method that is essentially identical to a reference method in all ways except for the identified difference. For example, a corresponding method expressing a nucleic acid encoding a transcriptional activator (e.g., Rtg1) would be the same in all aspects (e.g., genetic makeup of cell, temperature and time of culture, and so forth), except that the corresponding method would lack expression of the transcriptional activator (e.g., Rtg1).

In accordance with the present disclosure, there may be employed conventional molecular biology, microbiology, biochemical, and recombinant DNA techniques within the skill of the art. Such techniques are explained fully in the literature. The materials and methods of the disclosure will be further described in the following examples, which do not limit the scope of the methods and compositions of matter described in the claims.

### EXAMPLES

### Example 1: Rtg1 Overexpression Increased Expression of an Exogenous Protein

In this Example, an empty plasmid (Control) or a Rtg1 overexpression plasmid (pGAP-Rtg1 or pAOX1-Rtg1) was transformed into a P. pastoris strain that expressed red fluorescence protein (RFP) under an AOX1 promoter. Rtg1 was expressed under a constitutive GAP promoter (pGAP-Rtg1) or an inducible AOX1 promoter (pAOX1-Rtg1). Growth was carried out for 48 hours in YP media at 30° C. with dextrose and 300 ηg/ml Geneticin (G418). Fluorescence was measured using a fluorescence plate reader. Measurements were carried out with excitation at 520 nm and emission at 585 nm. A 50-fold dilution of the sample in water was made before measurements. As shown below in Table 4, Rtg1 expression led to 18-38% increase in RFP expression. Rtg1 overexpression from either pAOX1 or pGAP can lead to increased RFP expression indicating that the benefit can be achieved with or without a positive feedback loop, as Rtg1 overexpression under a non-mut promoter can also lead to increased RFP gene expression under a mut promoter.

TABLE 4

| | Normalized RFP |
|---|---|
| | fluorescence/OD600 |
| Control | 1.00 |
| pGAP-Rtg1 | 1.18 |
| pAOX1-Rtg1 | 1.38 |

### Example 2: Rtg1 Overexpression Increased Expression of an Exogenous Heme-Binding Protein

In this Example, an empty plasmid (Control) or a Rtg1 overexpression plasmid (pGAP-Rtg1 or pAOX1-Rtg1) was transformed in a P. pastoris strain that expressed the heme-binding protein leghemoglobin (LegH) and heme biosynthesis enzymes under an AOX1 promoter. Rtg1 was expressed under a constitutive GAP promoter (pGAP-Rtg1) or an inducible AOX1 promoter (pAOX1-Rtg1). Growth was carried out for 48 hours in YP media at 30° C. with dextrose and 300 μg/ml Geneticin (G418). LegH titer was measured by spectrophotometry of lysates purified by size-exclusion chromatography. A calibration curve was built with purified LegH using absorbance at 280 nm (for protein) and 415 nm (for heme). LegH titers of test samples were measured relative to the calibration sample. As shown below in Table 5, Rtg1 expression led to 16-19% increase in LegH titer. Details related to quantification of LegH are included below. Rtg1 overexpression from either pAOX1 or pGAP can lead to increased LegH expression indicating that the benefit can be achieved with or without a positive feedback loop, as Rtg1 overexpression under a non-mut promoter can also lead to increased LegH gene expression under a mut promoter.

TABLE 5

| | Normalized LegH titer |
|---|---|
| Control | 1.00 |
| pGAP-Rtg1 | 1.16 |
| pAOX1-Rtg1 | 1.19 |

LegH was quantified as described in U.S. Publication No. US20200340000A1, filed Apr. 24, 2020, which is incorporated herein by reference in its entirety. To initiate LegH quantification, cell broth samples were pelleted down (at 4000×g, 4° C., 30 min) and decanted. The pellet samples were then diluted four times with lysis buffer (150 mM NaCl, 50 mM Potassium Phosphate, pH 7.4). 300 μL of each resuspension was dispensed into a 96 well deep plate with 120 μL of beads (Zirconium/silica beads (0.5 mm)) per well for cell lysis. The lysis was done with a mini bead beater for 3 minutes, then the plate was cooled down on ice for 5 minutes, and followed with another 2 minutes of bead beating. The plate was then spun down (at 4000×g, 4° C., 30 min). The supernatant was filtered through a 0.2 μm filter plate (at 4000×g, 4° C., 60 min).

The filtered lysate was loaded onto a UHPLC with a size-exclusion column (Acquity BEH SEC column, 200 Å, 1.7 um, 4.6×150 mm). Method parameters: 1) Mobile phase: 5 mM NaCl, 50 mM Potassium Phosphate, (pH 7.4); 2) Flow rate: 0.3 mL/min; 3) Injection volume: 10 μL; 4) Run time: 15 min; 5) Sample tray temperature: 4° C. A calibration curve was built with a purified LegH standard using absorbance at 280 nm and 415 nm. The quantification was done using peak area with valley-to-valley peak integration method. The absorbance at 280 nm is proportional to the amount of the polypeptide present, and the absorbance at 415 nm is proportional to the amount of heme present. Where a peak is seen at the same elution time at both wavelengths, a heme containing protein is detected.

### Example 3: Rtg1 Overexpression Increased Expression of an Exogenous Protein

In this Example, an empty plasmid (Control) or a Rtg1 overexpression plasmid (pAOX1-Rtg1) was transformed in a P. pastoris strain that expressed bovine myoglobin (Mb) under an AOX1 promoter. Growth was carried out for 48 hours in YP media at 30° C. with dextrose and 300 μg/ml Geneticin (G418). A calibration curve was made using purified myoglobin. As shown below in Table 6, Rtg1 expression led to a 28% increase in Mb titer when expressed under an AOX1 promoter.

TABLE 6

| | Normalized Mb titer |
|---|---|
| Control | 1.00 |
| pAOX1-Rtg1 | 1.28 |

### Example 4: Rtg1 Overexpression Increased Expression of an Exogenous Protein Under Methanol Utilization (mut) Gene Promoters

In this Example, a cassette containing Rtg1 ORF along with an AOX1 promoter and terminator plasmid was integrated in a parent strain to obtain “Parent strain+Rtg1”. Plasmids containing green fluorescent protein (GFP) under mut gene promoters (AOX1, DAS1 and FLD1) were transformed in the parent strain and “Parent strain+Rtg1”. Growth was carried out for 48 hours in YP media at 30° C. with dextrose and 300 μg/ml Geneticin (G418). Fluorescence was measured using a fluorescence plate reader. Measurements were carried out with excitation at 485 nm and emission at 525 nm. A 50-fold dilution of the sample in water was made before measurements. Normalization was done by calculating GFP fluorescence/OD600 in “Parent strain+Rtg1” compared to the parent strain for the same promoter driving GFP expression. As shown below in Table 7, Rtg1 expression led to 11% to 98% increase in GFP expression depending on the promoter GFP was expressed from.

TABLE 7

| | | Normalized GFP |
|---|---|---|
| | GFP under promoter | fluorescence/OD600 |
| Parent strain | AOX1, DAS1, FLD1 | 1.00 |
| Parent strain + | AOX1 | 1.98 |
| Rtg1 | DAS1 | 1.18 |
| | FLD1 | 1.11 |

### Example 5: Rtg1 Overexpression Increased Expression of a Native Protein Under a Methanol Utilization (mut) Gene Promoter

In this Example, a cassette containing Rtg1 ORF along with an AOX1 promoter and terminator plasmid was integrated in a parent strain to obtain “Parent strain+Rtg1”. Growth was carried out for 48 hours in YP media at 30° C. with dextrose and 300 μg/ml Geneticin (G418). The protein level of AOX2, a protein in the methanol utilization (mut) pathway expressed under the AOX2 promoter, was monitored by Shotgun proteomics. As shown below in Table 8, Rtg1 expression led to a 189% increase in AOX2 expression.

TABLE 8

| | Normalized AOX2 |
|---|---|
| | Protein Level |
| Parent strain | 1.00 |
| Parent strain + Rtg1 | 2.89 |

### Example 6: Rtg1 and Mxr1 Overexpression Additively Increased Exogenous Protein Expression

In this Example, expression levels of green fluorescent protein (GFP) in “Parent strain+Rtg1” strain, “Parent strain+Mxr1” strain, and “Parent strain+Rtg1+Mxr1” strain were measured. “Parent strain+Rtg1” strain and “Parent strain+Mxr1” strain contained an exogenous copy of Rtg1 or Mxr1 under an AOX1 promoter in their genome, respectively. “Parent strain+Rtg1+Mxr1” strain contained a copy of both Rtg1 and Mxr1 under an AOX1 promoter in its genome. Plasmids containing GFP under an AOX1 promoter or DAS1 promoter were transformed in the parent strains and the daughter strains mentioned above. Growth was carried out for 48 hours in YP media at 30° C. with dextrose and 300 μg/ml Geneticin (G418). Normalization was done by calculating GFP fluorescence/OD600 in each daughter strain compared to the parent strain for the same promoter driving GFP expression.

As shown below in Table 9, Rtg1 and Mxr1 overexpression led to an increase of 70% and 252% in AOX1 promoter driven GFP expression individually and to an increase of 472% in GFP expression when combined compared to the parent strain. Similarly, Rtg1 and Mxr1 overexpression led to an increase of 15% and 108% in DAS1 promoter driven GFP expression individually and to an increase of 251% in GFP expression when combined compared to the parent strain.

TABLE 9

| Promoter driving | | Normalized GFP |
|---|---|---|
| GFP expression | Strain | fluorescence/OD600 |
| AOX1 | Parent strain | 1.00 |
| | Parent strain + Rtg1 | 1.75 |
| | Parent strain + Mxr1 | 3.52 |
| | Parent strain + Rtg1 + Mxr1 | 5.72 |
| DAS1 | Parent strain | 1.00 |
| | Parent strain + Rtg1 | 1.15 |
| | Parent strain + Mxr1 | 2.08 |
| | Parent strain + Rtg1 + Mxr1 | 3.51 |

### Example 7: Rtg2 Overexpression Increased Exogenous Protein Expression

In this Example, a cassette containing Rtg2 ORF along with an AOX1 promoter and terminator plasmid was integrated in a parent strain to obtain “Parent strain+Rtg2”. Plasmids containing green fluorescent protein (GFP) under an AOX1 promoter were transformed in the parent strain and “Parent strain+Rtg2”. Growth was carried out for 48 hours in YP media at 30° C. with dextrose and 300 μg/ml Geneticin (G418). Normalization was done by calculating GFP fluorescence/OD600 in “Parent strain+Rtg2” compared to the parent strain. As shown below in Table 10, Rtg2 expression led to a 40% increase in GFP expression.

TABLE 10

| | | Normalized GFP |
|---|---|---|
| | GFP under promoter | fluorescence/OD600 |
| Parent strain | AOX1 | 1.00 |
| Parent strain + Rtg2 | | 1.40 |

### Example 8: Mxr1, Rtg1, and Rtg2 Overexpression Increased Exogenous Protein Expression

In this Example, cassettes containing Rtg1, Rtg2, and/or Mxr1 along with AOX1 promoter and terminator plasmid were integrated in a parent strain. Plasmids containing green fluorescent protein (GFP) under an AOX1 promoter were transformed in each strain. Growth was carried out for 48 hours in YP media at 30° C. with dextrose and 300 μg/ml Geneticin (G418). Normalization was done by calculating GFP fluorescence/OD600 in each strain compared to the parent strain. As shown below in Table 11, Mxr1, Rtg1, and Rtg2 expression led to greater than a 500% increase in GFP expression.

TABLE 11

| | Normalized GFP |
|---|---|
| | fluorescence/OD600 |
| Parent strain | 1.00 |
| Parent strain + Rtg1 | 1.63 |
| Parent strain + Rtg2 | 1.40 |
| Parent strain + Rtg1 + Rtg2 | 1.90 |
| Parent strain + Mxr1 + Rtg1 | 5.69 |
| Parent strain + Mxr1 + Rtg1 + Rtg2 | 6.22 |
| Parent strain + Mxr1 + Rtg1 + Rtg2 | 6.19 |

### Example 9: Rtg1 and Mit1 or Trm1 Overexpression Increased Exogenous Protein Expression

In this Example, cassettes containing Rtg1 with Mit1 or Trm1 along with AOX1 promoter and terminator plasmid were integrated in a parent strain. Plasmids containing green fluorescent protein (GFP) under an AOX1 promoter were transformed in each strain. Growth was carried out for 48 hours in YP media at 30° C. with dextrose and 300 μg/ml Geneticin (G418). Normalization was done by calculating GFP fluorescence/OD600 in each strain compared to the parent strain. As shown below in Table 12, Mit1 alone or in combination with Rtg1 led to greater than a 900% increase in GFP expression. As also shown in Table 12, the combination of Mxr1 and Rtg1 with or without Trm1 led to at least a 600% increase in GFP expression.

TABLE 12

| | Normalized GFP |
|---|---|
| | fluorescence/OD600 |
| Parent strain | 1.00 |
| Parent strain + Rtg1 | 1.5 |
| Parent strain + Trm1 | 1.6 |
| Parent strain + Mit1 | 9.1 |
| Parent strain + Mxr1 | 2.9 |
| Parent strain + Trm1 + Rtg1 | 2.9 |
| Parent strain + Mit1 + Rtg1 | 16.8 |
| Parent strain + Mxr1 + Rtg1 | 6.0 |
| Parent strain + Mxr1 + Rtg1 + Trm1 | 7.1 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A yeast cell comprising:
a first exogenous nucleic acid encoding a retrograde regulation protein (Rtg) operably linked to a first promoter element, and
a second exogenous nucleic acid encoding a polypeptide operably linked to the first promoter element or a second promoter element.

2. The yeast cell of claim 1, wherein the Rtg is Rtg1 or Rtg2 from Pichia pastoris or Saccharomyces cerevisiae.

3. The yeast cell of claim 1, wherein the polypeptide is selected from the group consisting of an antibody or fragment thereof, an enzyme, a regulatory protein, a peptide hormone, a blood clotting protein, a cytokine, a cytokine inhibitor, and a heme-binding protein.

4. The yeast cell of claim 3, wherein the heme-binding protein is selected from the group consisting of a globin, a cytochrome, a cytochrome c oxidase, a ligninase, a catalase, and a peroxidase.

5. The yeast cell of claim 1, wherein the first exogenous nucleic acid, the second exogenous nucleic acid, or both the first exogenous nucleic acid and the second exogenous nucleic acid is stably integrated into the genome of the yeast cell.

6. The yeast cell of claim 1, wherein the first exogenous nucleic acid, the second exogenous nucleic acid, or both the first exogenous nucleic acid and the second exogenous nucleic acid is extrachromosomally expressed from a replication-competent plasmid.

7. The yeast cell of claim 1, wherein the first promoter element is a constitutive promoter element.

8. The yeast cell of claim 1, wherein the first promoter element, the second promoter element, or both the first promoter element and the second promoter element is an inducible promoter element.

9. The yeast cell of claim 8, wherein the inducible promoter element is a methanol-inducible promoter element.

10. The yeast cell of claim 9, wherein the methanol-inducible promoter element is selected from the group consisting of an alcohol oxidase 1 (AOX1) promoter element from Pichia pastoris, an alcohol oxidase 2 (AOX2) promoter element from Pichia pastoris, a catalase 1 (CAT1) promoter from P. pastoris, a formate dehydrogenase (FMD) promoter from Hansenula polymorpha, an AOD1 promoter element from Candida boidinii, a FGH promoter element from Candida boidinii, a MOX promoter element from Hansenula polymorpha, a MODI promoter element from Pichia methanolica, a DHAS promoter element from Pichia pastoris, a FLD1 promoter element from Pichia pastoris, and a PEX8 promoter element from Pichia pastoris.

11. The yeast cell of claim 1, further comprising a third exogenous nucleic acid encoding a transcriptional activator selected from methanol expression regulator 1 (Mxr1), methanol-induced transcription factor 1 (Mit1), and Trm1 operably linked to the first promoter element, the second promoter element, or a third promoter element.

12. (canceled)

13. The yeast cell of claim 11, wherein the third promoter element is a constitutive promoter element or a methanol-inducible promoter element.

14. A yeast cell comprising:
a first exogenous nucleic acid encoding a first transcriptional activator selected from Rtg1, Rtg2, Mxr1, Mit1, and Trm1 operably linked to a first promoter element,
a second exogenous nucleic acid encoding a second transcriptional activator selected from Rtg1, Rtg2, Mxr1, Mit1, and Trm1 operably linked to the first promoter element or a second promoter element, wherein the first transcriptional activator and the second transcriptional activator are different, and
a third exogenous nucleic acid encoding a polypeptide operably linked to the first promoter element, the second promoter element, or a third promoter element.

15. The yeast cell of claim 14, further comprising a fourth exogenous nucleic acid encoding one or more heme biosynthesis enzymes operably linked to the first promoter element, the second promoter element, the third promoter element, or a fourth promoter element.

16. The yeast cell of claim 15, wherein the one or more heme biosynthesis enzymes are selected from the group consisting of glutamate-1-semialdehyde (GSA) aminotransferase, 5-aminolevulinic acid (ALA) synthase, ALA dehydratase, porphobilinogen (PBG) deaminase, uroporphyrinogen (UPG) III synthase, UPG III decarboxylase, coproporphyrinogen (CPG) III oxidase, protoporphyrinogen (PPG) oxidase, and ferrochelatase.

17. The yeast cell of claim 15, wherein the fourth promoter element is a constitutive promoter element or a methanol-inducible promoter element.

18. The yeast cell of claim 1, wherein the yeast cell is a methylotrophic yeast cell or a non-methylotrophic yeast cell.

19. The yeast cell of claim 18, wherein the methylotrophic yeast cell is a Pichia cell.

20. (canceled)

21. A method for expressing a polypeptide, the method comprising:
providing the yeast cell of claim 1, and
culturing the yeast cell under conditions suitable for expression of the first and the second exogenous nucleic acids.

22. (canceled)

23. (canceled)

24. A method for expressing a polypeptide, the method comprising:
providing the yeast cell of claim 14, and
culturing the yeast cell under conditions suitable for expression of the first, second, and third exogenous nucleic acids.


SEQUENCE LISTING (44 sequences)

SEQ ID NO: 1 - DNA, 741 bp, Pichia pastoris
  source 1..741: mol_type = other DNA
atggatagtaatcaatggcccaaggcggagcgtccgttccaagaaaatgaaatcttggacttttccagcttggataatatactcgacactgatactgaatttggaagaagtaccagtaaacatgtacaacacacagaccccccactgcaacaggaccagttgctgacatacaatatagaccaggcgtcacaaaatactccctctcctaacttctatccttcaagcattgatgttaagcagtctctttcaaaggctttacccgcctcgcataatgtcaagtccgaatctccacaacaggccgagtacaacagcaatgaggattccaacaatcaatccgaatcaaatataaatacagcgaagtcccggaggagctcagtggtgacaactccaggtgggactattgttgagcgcaagcgcagagacaatatcaatgaacgtatacaggacctactcactgttattccggagtcttttttcctagaccccaaggataaagcaaaagctacaggtaccaaagatggaaagcctaataaggggcaaattttaacaaaagcagtagagtatattcattgtcttcaacaggatattgacgatagaaaccgtcaagaggtcgctttgtccttgaaactcaaaaacttagagattgctcataatgtaccggaagaacgcagagaagatttaaaaaatacctctgccgaaaagggcctgggtagcattggtgttggaccactagcagattga

SEQ ID NO: 2 - protein, 246 aa, Pichia pastoris
MDSNQWPKAERPFQENEILDFSSLDNILDTDTEFGRSTSKHVQHTDPPLQQDQLLTYNIDQASQNTPSPNFYPSSIDVKQSLSKALPASHNVKSESPQQAEYNSNEDSNNQSESNINTAKSRRSSVVTTPGGTIVERKRRDNINERIQDLLTVIPESFFLDPKDKAKATGTKDGKPNKGQILTKAVEYIHCLQQDIDDRNRQEVALSLKLKNLEIAHNVPEERREDLKNTSAEKGLGSIGVGPLAD

SEQ ID NO: 3 - DNA, 741 bp, Pichia pastoris
  source 1..741: mol_type = other DNA
atggatagtaatcaatggcccaaggcggagcgtccgttccaagaaaatgaaatcttggacttttccagcttggataatatactcgacactgatactgaatttggaagaagtaccagtaaacatgtacaacacacagaccccccactgcaacaggaccagttgctgacatacaatatagaccaggcgtcacaaaatactccctctcctaacttctatccttcaagcattgatgttaagcagtctctttcaaaggctttacccgcctcgcataatgtcaagtccgaatctccacaacaggccgagtacaacagcaatgaggattccaacaatcaatccgaatcaaatataaatacagcgaagtcccggaggagctcagtggtgacaactccaggtgggactattgttgagcgcaagcgcagagacaatgtcaatgaacgtatacaggacctactcactgttattccggagtcttttttcctagaccccaaggataaagcaaaagctacaggtaccaaagatggaaagcctaataaggggcaaattttaacaaaagcagtagagtatattcattgtcttcaacaggatattgacgatagaaaccgtcaagaggtcgctttgtccttgaaactcaaaaacttagagattgctcataatgtaccggaagaacgcagagaagatttaaaaaatacctctgccgaaaagggcctgggtagcattggtgttggaccactagcagattga

SEQ ID NO: 4 - protein, 246 aa, Pichia pastoris
MDSNQWPKAERPFQENEILDFSSLDNILDTDTEFGRSTSKHVQHTDPPLQQDQLLTYNIDQASQNTPSPNFYPSSIDVKQSLSKALPASHNVKSESPQQAEYNSNEDSNNQSESNINTAKSRRSSVVTTPGGTIVERKRRDNVNERIQDLLTVIPESFFLDPKDKAKATGTKDGKPNKGQILTKAVEYIHCLQQDIDDRNRQEVALSLKLKNLEIAHNVPEERREDLKNTSAEKGLGSIGVGPLAD

SEQ ID NO: 5 - DNA, 1713 bp, Pichia pastoris
  source 1..1713: mol_type = other DNA
atgtccacagtagagctacaggcaaatgaagctgaaatagtagcacgctcattggtagccatcgtggacattggttccaatggaatcaggttttctgtgtcttccaccgcctcccatcatgccagaattatgccttgtgtcttcaaggacagattgggtatttcactgttcgacgcccaactcgacaagggctctgccagttctatcagcacacgtaagccgataccccaggaagcaatcactgagatctgtttggccatgaaacgattccagttgatttgtgaggattttggagtttcaaatgataacgtgaagatagttgcaacagaagcaactagggaagccccaaactctaaagaattcagggacgcaattgcgaagaccacaggatgggaagttgaattgctttcaaaggaagacgagggccgatgcggtgctttcggcgttgcctcctcattccataatatctctggtatcttcatggatgtggggggaggatctactcagctgagctgggtatccacagtcaatggggatgtcagacttgctgaataccctatatctctaccttatggggctgctgcccttactcagcgattattatatgaagatgaaagagaggtttatgaagaggttcgtcaggcttatgaattagcgttggagaagataaaaattcctacagagctcatcgaagaagctgaaaaaaatggcggatttaatttgtatacttgcggcggaggattccgcggtgtgggacatcttcttcttcatgaagacccaaactatccaattcaaacgatcatcaatggttacacaactggcttcaagaaagtcgaattgttggcgaactaccttttgttgaagaaagaagttccaaacttcagtgaaggaagcccaaagattttcagagtttcagaaagacgaaaacaacagctccctgctgttggactactgatgagtgcagcattccaagtgttaccaaaaattagaactgtcagtttcagtgaaggcggtgtacgtgagggtgtattgtacagtagaatctcaccatctataagatctgaagatccccttttgactgccactcgtccttatgctccccttttgtctgagcaatacaggaaacttcttctcggtgcacttccagaggaagtcccctccgagatcacccagacgatagtacctgctctttgcaacattgcatttgtccactgttcatatcctaaagagttgcaaccaacagcagcgctccacatggctacctctggtattatcgctggaactcatggcctttctcacaaagtgcgtgctctaataggcctagcatgttgtgaacgttgggggtttgatcttcctgaatcagaagaagttttttacggcaaactagaaaaattggttattcaatcagatccaaatgacggtgaaaggttactatactggacaaaatattgtggaaaaataatgtttgttatttgcggagtacatcccggaggaaacatacgtccaggtgttatagacttcaacgtaataccgcgggcagaagcaaacaagaccaacacggctgttcaagtgggaatgtcagccaatgatgtcaaatcgagttacactgttaagaacagaattgccagtttacaacgaaaaatcaagaaactgaacaaatcttacaaaggaaaagacagagttgtggtagaggttgagtatagaatgtcatag

SEQ ID NO: 6 - protein, 570 aa, Pichia pastoris
MSTVELQANEAEIVARSLVAIVDIGSNGIRFSVSSTASHHARIMPCVFKDRLGISLFDAQLDKGSASSISTRKPIPQEAITEICLAMKRFQLICEDFGVSNDNVKIVATEATREAPNSKEFRDAIAKTTGWEVELLSKEDEGRCGAFGVASSFHNISGIFMDVGGGSTQLSWVSTVNGDVRLAEYPISLPYGAAALTQRLLYEDEREVYEEVRQAYELALEKIKIPTELIEEAEKNGGFNLYTCGGGFRGVGHLLLHEDPNYPIQTIINGYTTGFKKVELLANYLLLKKEVPNFSEGSPKIFRVSERRKQQLPAVGLLMSAAFQVLPKIRTVSFSEGGVREGVLYSRISPSIRSEDPLLTATRPYAPLLSEQYRKLLLGALPEEVPSEITQTIVPALCNIAFVHCSYPKELQPTAALHMATSGIIAGTHGLSHKVRALIGLACCERWGFDLPESEEVFYGKLEKLVIQSDPNDGERLLYWTKYCGKIMFVICGVHPGGNIRPGVIDFNVIPRAEANKTNTAVQVGMSANDVKSSYTVKNRIASLQRKIKKLNKSYKGKDRVVVEVEYRMS

SEQ ID NO: 7 - DNA, 3468 bp, Pichia pastoris
  source 1..3468: mol_type = other DNA
atgagcaatctacccccaacttttggttccactagacaatctccagaagaccaatcacctcccgtgcccaaggagctgtcattcaatgggaccacaccctcaggaaagctacgcttatttgtctgtcagacatgtactcgagcatttgctcgtcaggaacacttgaaacgacacgaaaggtctcacaccaaggagaaacctttcagctgcggcatttgttctcgtaaattcagccgtcgagatctgttattgagacatgcccaaaaactgcacagcaactgctctgatgcggccataacaagactaaggcgcaaggcaactcgtcggtcttctaatgccgcgggttccatatctggttctactccggtgacaacgccaaatactatgggtacgcccgaagatggcgagaaacgaaaagttcagaaactggccggccgccgggactcaaatgaacagaaactgcaactgcaacaacaacatctacagcaacaaccacagttgcaataccaacaatctcttaagcagcatgaaaatcaagtccagcagcctgatcaagatccattgatatccccgagaatgcaattattcaatgattccaaccatcacgtaaacaatttgtttgatcttggactaagaagagcttccttctccgccgttagtggaaataattatgcccattatgtgaataattttcaacaagatgcctcttctaccaatccaaatcaagattcaaataatgccgaatttgagaatattgaattttctaccccacaaatgatgcccgttgaagatgctgaaacttggatgaacaacatgggtccaattccgaacttctctctcgatgtgaacaggaacattggtgatagctttacagatatacaacacaagaattcagagcctattatatccgaaccgcccaaggacaccgctccaaacgacaagaagttgaatggctactctttttacgaagcccccatcaagccattagaatccctattttctgtcaggaatacaaagagaaacaagtataaaacaaatgacgactctccagacaccgtggataataactccgcaccggctgctaataccattcaagaacttgagtcttctttgaatgcatccaagaatttttgcttgccaactggttattccttctatggtaatttggaccaacagactttctctaacacgttatcatgcacttcttctaatgccacaatttcgcccattctactcgataactccattaataataactccactagtgacgtgagaccagaatttagaacacaaagtgtcacctctgaaatgagtcaagcccctccccctcctcaaaaaaacaactcgaaatattccaccgaagttctttttaccagcaacatgcggtcgtttattcactacgctctttccaagtatccttttattggtgtgcccactccaactcttccggagaacgaaagactaaatgaatatgctgattcattcaccaaccgtttcttaaatcattatcctttcatacatgtcacgattctcaaagaatactcccttttcaaggcaattttagatgagaatgagtcgactaagaactgggaaaataatcagttttacttagagaaccaacgaatatcaattgtttgtcttcctcttttggtggctacgataggtgcagtactatcaaacaacaaaaaggatgcttcgaatttatacgaagcttcaaggcgttgtattcatgtttacttagattccaggaaaaagatacccacttccttgtccgcaaataacaatgactctccactttggctaattcaatccctgacgttatctgttatgtatgggttatttgcggacaatgacattagtttgaatgtcgtgatcagacaagttaacgcacttaattctctggtcaagacttcgggcctgaataggacctcaattatagatcttttcaacatcaacaaacctttggataatgaactctggaatcaattcgtgaaaatagagtccaccgtaaggacaatccacacgatttttcaaatcagttccaacttaagcgccttgtacaatattattccatcgttgaaaattgatgacctaatgattactctaccagttcccacaacactttggcaagctgattcttttgtgaaattcaaaagtctaagttacggaaatcagatcccttttcaatatacaagagtactacagaatttgattgattacaatcagccattgagcgatggaaaatttttgtatgaaaaccatgtaagtgagtttggactcatatgcctacagaatggtctacaccaatacagctatttccaaaaattgactgctgtcaataacagagaagatgcgctattcacaaaggttgttaattcacttcacagttgggataggatgatttcgaattctgatttgtttccaaagaagatatatcagcagagttgcttgattttggactcaaagttgcttaataatttcctgattgtcaagagctcattgaaagtttcgaccggagacgttagttctttgaataagttaaaagaaaacgtgtggcttaaaaactggaatcaagtgtgtgctatctattataacagcttcatgaacattcctgctcccagtattcaaaagaagtacaatgacatagagtttgtggatgacatgattaatttgagtctaatcatcatcaagattatgaaactcattttctataacaatgtcaaagacaattatgaggatgaaaatgacttcaaattgcaagagttaaatttaacatttgacaattttgatgagaaaatatccttgaatttgacaatattattcgatatatttttgatgatctacaagataattaccaattacgaaaagtttatgaagatcaaacacaagtttaattactacaattctaattcgaatataagcttcttgcatcatttcgaactctcctcggttatcaataacacccaaatgaaccagaatgattatatgaaaacagatattgatgaaaagcttgatcagcttttccacatctatcaaacatttttccggctgtatctggatttagaaaagtttatgaagttcaaattcaactatcatgactttgagacagagttttcaagtctctcaatatccaatatactgaacactcatgctgcttctaacaatgacacaaatgctgctgatgctatgaatgccaaggatgaaaaaatatctcccacaactttgaatagcgtattacttgctgatgaaggaaatgaaaattccggtcgtaataacgattcagaccgcctgttcatgctgaacgagctaattaattttgaagtaggtttgaaatttctcaagataggtgagtcattttttgatttcttgtatgagaataactacaagttcatccacttcaaaaacttaaatgacggaatgttccacatcaggatatacctagaaaaccgactagatggtggtgtctag

SEQ ID NO: 8 - protein, 1155 aa, Pichia pastoris
MSNLPPTFGSTRQSPEDQSPPVPKELSFNGTTPSGKLRLFVCQTCTRAFARQEHLKRHERSHTKEKPFSCGICSRKFSRRDLLLRHAQKLHSNCSDAAITRLRRKATRRSSNAAGSISGSTPVTTPNTMGTPEDGEKRKVQKLAGRRDSNEQKLQLQQQHLQQQPQLQYQQSLKQHENQVQQPDQDPLISPRMQLFNDSNHHVNNLFDLGLRRASFSAVSGNNYAHYVNNFQQDASSTNPNQDSNNAEFENIEFSTPQMMPVEDAETWMNNMGPIPNFSLDVNRNIGDSFTDIQHKNSEPIISEPPKDTAPNDKKLNGYSFYEAPIKPLESLFSVRNTKRNKYKTNDDSPDTVDNNSAPAANTIQELESSLNASKNFCLPTGYSFYGNLDQQTFSNTLSCTSSNATISPILLDNSINNNSTSDVRPEFRTQSVTSEMSQAPPPPQKNNSKYSTEVLFTSNMRSFIHYALSKYPFIGVPTPTLPENERLNEYADSFTNRFLNHYPFIHVTILKEYSLFKAILDENESTKNWENNQFYLENQRISIVCLPLLVATIGAVLSNNKKDASNLYEASRRCIHVYLDSRKKIPTSLSANNNDSPLWLIQSLTLSVMYGLFADNDISLNVVIRQVNALNSLVKTSGLNRTSIIDLFNINKPLDNELWNQFVKIESTVRTIHTIFQISSNLSALYNIIPSLKIDDLMITLPVPTTLWQADSFVKFKSLSYGNQIPFQYTRVLQNLIDYNQPLSDGKFLYENHVSEFGLICLQNGLHQYSYFQKLTAVNNREDALFTKVVNSLHSWDRMISNSDLFPKKIYQQSCLILDSKLLNNFLIVKSSLKVSTGDVSSLNKLKENVWLKNWNQVCAIYYNSFMNIPAPSIQKKYNDIEFVDDMINLSLIIIKIMKLIFYNNVKDNYEDENDFKLQELNLTFDNFDEKISLNLTILFDIFLMIYKIITNYEKFMKIKHKFNYYNSNSNISFLHHFELSSVINNTQMNQNDYMKTDIDEKLDQLFHIYQTFFRLYLDLEKFMKFKFNYHDFETEFSSLSISNILNTHAASNNDTNAADAMNAKDEKISPTTLNSVLLADEGNENSGRNNDSDRLFMLNELINFEVGLKFLKIGESFFDFLYENNYKFIHFKNLNDGMFHIRIYLENRLDGGV

SEQ ID NO: 9 - DNA, 2667 bp, Pichia pastoris
  source 1..2667: mol_type = other DNA
atgagtaccgcagccccaatcaaggaagaaagccaatttgcccatttgaccctaatgaacaaggatataccttcgaacgcaaaacaggcaaagtcgaaagtttcagcggcccctgctaagacgggctccaaatctgctggtggatctggcaacaacaacgctgcacctgtgaaaaaaagagtccgcacgggctgtttgacctgccgaaagaagcacaagaaatgtgacgagaacagaaacccaaaatgtgacttttgcactttgaaaggcttggaatgtgtctggccagagaacaataagaagaatatcttcgttaacaactccatgaaggatttcttaggcaagaaaacggtggatggagctgatagtctcaatttggccgtgaatctgcaacaacagcagagttcaaacacaattgccaatcaatcgctttcctcaattggattggaaagttttggttacggctctggtatcaaaaacgagtttaacttccaagacttgataggttcaaactctggcagttcagatccgacattttcagtagacgctgacgaggcccaaaaactcgacatttccaacaagaacagtcgtaagagacagaaactaggtttgctgccggtcagcaatgcaacttcccatttgaacggtttcaatggaatgtccaatggaaagtcacactctttctcttcaccgtctgggactaatgacgatgaactaagtggcttgatgttcaactcaccaagcttcaaccccctcacagttaacgattctaccaacaacagcaaccacaatataggtttgtctccgatgtcatgcttattttctacagttcaagaagcatctcaaaaaaagcatggaaattccagtagacacttttcatacccatctgggccggaggacctttggttcaatgagttccaaaaacaggccctcacagccaatggagaaaatgctgtccaacagggagatgatgcttctaagaacaacacagccattcctaaggaccagtcttcgaactcatcgattttcagttcacgttctagtgcagcttctagcaactcaggagacgatattggaaggatgggcccattctccaaaggaccagagattgagttcaactacgattcttttttggaatcgttgaaggcagagtcaccctcttcttcaaagtacaatctgccggaaactttgaaagagtacatgacccttagttcgtctcatctgaatagtcaacactccgacactttggcaaatggcactaacggtaactattctagcaccgtttccaacaacttgagcttaagtttgaactccttctctttctctgacaagttctcattgagtccaccaacaatcactgacgccgaaaagttttcattgatgagaaacttcattgacaacatctcgccatggtttgacacttttgacaataccaaacagtttggaacaaaaattccagttctggccaaaaaatgttcttcattgtactatgccattctggctatatcttctcgtcaaagagaaaggataaagaaagagcacaatgaaaaaacattgcaatgctaccaatactcactacaacagctcatccctactgttcaaagctcaaataatattgagtacattatcacatgtattctcctgagtgtgttccacatcatgtctagtgaaccttcaacccagagggacatcattgtgtcattggcaaaatacattcaagcatgcaacataaacggatttacatctaatgacaaactggaaaagagtattttctggaactatgtcaatttggatttggctacttgtgcaatcggtgaagagtcaatggtcattccttttagctactgggttaaagagacaactgactacaagaccattcaagatgtgaagccatttttcaccaagaagactagcacgacaactgacgatgacttggacgatatgtatgccatctacatgctgtacattagtggtagaatcattaacctgttgaactgcagagatgcgaagctcaattttgagcccaagtgggagtttttgtggaatgaactcaatgaatgggaattgaacaaacccttgacctttcaaagtattgttcagttcaaggccaatgacgaatcgcagggcggatcaacttttccaactgttctattctccaactctcgaagctgttacagtaaccagctgtatcatatgagctacatcatcttagtgcagaataaaccacgattatacaaaatcccctttactacagtttctgcttcaatgtcatctccatcggacaacaaagctgggatgtctgcttccagcacacctgcttcagaccaccacgcttctggtgatcatttgtctccaagaagtgtagagccctctctttcgacaacgttgagccctccgcctaatgcaaacggtgcaggtaacaagttccgctctacgctctggcatgccaagcagatctgtgggatttctatcaacaacaaccacaacagcaatctagcagccaaagtgaactcattgcaaccattgtggcacgctggaaagctaattagttccaagtctgaacatacacagttgctgaaactgttgaacaaccttgagtgtgcaacaggctggcctatgaactggaagggcaaggagttaattgactactggaatgttgaagaatag

SEQ ID NO: 10 - protein, 888 aa, Pichia pastoris
MSTAAPIKEESQFAHLTLMNKDIPSNAKQAKSKVSAAPAKTGSKSAGGSGNNNAAPVKKRVRTGCLTCRKKHKKCDENRNPKCDFCTLKGLECVWPENNKKNIFVNNSMKDFLGKKTVDGADSLNLAVNLQQQQSSNTIANQSLSSIGLESFGYGSGIKNEFNFQDLIGSNSGSSDPTFSVDADEAQKLDISNKNSRKRQKLGLLPVSNATSHLNGFNGMSNGKSHSFSSPSGTNDDELSGLMFNSPSFNPLTVNDSTNNSNHNIGLSPMSCLFSTVQEASQKKHGNSSRHFSYPSGPEDLWFNEFQKQALTANGENAVQQGDDASKNNTAIPKDQSSNSSIFSSRSSAASSNSGDDIGRMGPFSKGPEIEFNYDSFLESLKAESPSSSKYNLPETLKEYMTLSSSHLNSQHSDTLANGTNGNYSSTVSNNLSLSLNSFSFSDKFSLSPPTITDAEKFSLMRNFIDNISPWFDTFDNTKQFGTKIPVLAKKCSSLYYAILAISSRQRERIKKEHNEKTLQCYQYSLQQLIPTVQSSNNIEYIITCILLSVFHIMSSEPSTQRDIIVSLAKYIQACNINGFTSNDKLEKSIFWNYVNLDLATCAIGEESMVIPFSYWVKETTDYKTIQDVKPFFTKKTSTTTDDDLDDMYAIYMLYISGRIINLLNCRDAKLNFEPKWEFLWNELNEWELNKPLTFQSIVQFKANDESQGGSTFPTVLFSNSRSCYSNQLYHMSYIILVQNKPRLYKIPFTTVSASMSSPSDNKAGMSASSTPASDHHASGDHLSPRSVEPSLSTTLSPPPNANGAGNKFRSTLWHAKQICGISINNNHNSNLAAKVNSLQPLWHAGKLISSKSEHTQLLKLLNNLECATGWPMNWKGKELIDYWNVEE

SEQ ID NO: 11 - DNA, 2970 bp, Pichia pastoris
  source 1..2970: mol_type = other DNA
atgcctcctaaacatcggctggagcagagtatacagcccatggcttctcaacaaatagtacccggtaataaggttattctgccgaatccaaaagtagatgcaaaatctaccccaaacatttcagttcagaagagaagaagagtcaccagagcttgtgatgaatgtcggaaaaagaaggtcaaatgtgatggtcaacaaccatgcattcattgtaccgtttattcctatgagtgcacttacagccaaccttccagtaagaagagacagggacaatctctgagtctgagtgctccgtcaaacattaatgcaacaagttccgtacaaaaatctgtaaaacctcctgaaatcgatttccaaaggatgagagacgcactcaaatattacgaagatcttttaaaccagttgatataccccaacagtgctccaactgttcgagttaatccgattcgtctagcatcgatcttaaaacaattgagagccgataaatcaagtgatgaattaatttcagtcaaggctctttctgacaattacattgagatgcttcacaaaacgatgcaacaacctgtacagcagccagctcctccttcattggggcaaggagggtccttctctaatcacagtcccaatcataataatgcttctattgatggttccatagaatctaatctagggagggaaatacgtatcatattacctccgagagatattgcgctgaagcttatctacaagacttgggacaacgcgtgtgtacttttccgcttttatcacagacccgcatttattgaggacctgaatgagttatatgaaacagatttggcaaactacaccaataaacaacaaaggtttttacctcttgtatattcggtgatggcttgtggtgctcttttttgcaagactgatgggattaatcacggccaaaagagctccaagcccaaagactcttctgatgaaagtctcatagacgatgagggttacaagtattttattgccgcaagaaaactaatagatatcacggataccagggatacctacggaattcagactattgttatgctgatcatttttttacaatgttcggctcgtctttcaacatgctattcttatattggcattgctctaagagctgcattgagagaaggtttgcatcgtcagttgaactatcctttcaatccaattgagttagaaacaagaaagcgtcttttttggactatctataaaatggacatctatgtcaatacaatgctggggcttccaagaaccatttctgaagaggatttcgaccaggaaatgcctatcgaacttgatgatgagaacattagtgaaaccggatataggttcgatttacaaggtacaaagttatccagttcaggaatagccaatgctcacactagattgatattcataatgaagaaaattgtgaaaaaattatatcctgtcaaactacagaaaccaacctcaaacagtggcgataccccacttgagaacaatgatttattggctcatgaaatcgttcatgaacttgagatggatctccaaaattgggtcaatagtctacctgcagaactaaaaccggggatagaaccaccgaccgagtattttaaagctaacagattgcttcatttggcatacctgcatgtcaagattattctctacaggccatttattcattacatctcagaaaaggataaggttggaaatagttctatccctccgtcgcccgaagagatcacttctatcgagaaagccaagaattgtgtcaatgttgccagaattgttgttaaactagccgaagacatgattaataggaaaatgttaagtggttcatattggttttccatttataccatttttttttccgtggcatgtctggtgtactatgttcatttcgctccaccgaagaaagacaatggagaactggatccccaatacatggaaatcaagaaagatacagagagtggaagagaggtcttaaatatcctcaaagatagtagtatggcggcaagaagaacgtataatattctcaactctttgtttgagcagttaaacagaagaactgcaaaggtcaacctagcaaaggcacagcaaccaccatcagggttgaataacccagctgctacccagtatcagaaacagggtgaacacaggcagttacaaccaagtaactattctggaactgtgaaatctgtggacccagagaatatcgattactcttcctttggttctcagtttgaaaacactaacatcgaagatggttcctcaaatacaaagattgatcagaaagtgaatggggtgaactacatcgatggtgtgtttacagggatcaacctaaatatgcctaatctctcagaaacttctaacactcaaggtatcgataatccagcatttcaaagtataaacaattctaatttgaacaataattttgtacaaacaaagtacattcccggcatgatggaccagctagatatgaaaattttcggaagattccttccaccttacatgctgaactccaacaaggttgaacagggacaaaatgaaaggaacctatcaggccaaccatcctcgtcgaatactcctgatggatcacaacctgtgacagttctggatggattatacccgttgcagaatgataataataataaccacgacccaggaaattcaaagtctgttgtaaataacagtaactcggtagaaaacttactacagaactttacaatggtgccctcggggttgtcatcaacagtgcaaaatcctgaagcggcccaaaaattcaataatcatatgtcaaacatatcgaatatgaatgatccaagaagagctagcgtagctacatcagatggatccaatgacatggatcatcatagccaaggcccgataaacaaagatttgaaaccgttgagcaactacgagtttgacgatctcttctttaatgattggaccactgcgccagatacaataaattttgacagttaa

SEQ ID NO: 12 - protein, 989 aa, Pichia pastoris
MPPKHRLEQSIQPMASQQIVPGNKVILPNPKVDAKSTPNISVQKRRRVTRACDECRKKKVKCDGQQPCIHCTVYSYECTYSQPSSKKRQGQSLSLSAPSNINATSSVQKSVKPPEIDFQRMRDALKYYEDLLNQLIYPNSAPTVRVNPIRLASILKQLRADKSSDELISVKALSDNYIEMLHKTMQQPVQQPAPPSLGQGGSFSNHSPNHNNASIDGSIESNLGREIRIILPPRDIALKLIYKTWDNACVLFRFYHRPAFIEDLNELYETDLANYTNKQQRFLPLVYSVMACGALFCKTDGINHGQKSSKPKDSSDESLIDDEGYKYFIAARKLIDITDTRDTYGIQTIVMLIIFLQCSARLSTCYSYIGIALRAALREGLHRQLNYPFNPIELETRKRLFWTIYKMDIYVNTMLGLPRTISEEDFDQEMPIELDDENISETGYRFDLQGTKLSSSGIANAHTRLIFIMKKIVKKLYPVKLQKPTSNSGDTPLENNDLLAHEIVHELEMDLQNWVNSLPAELKPGIEPPTEYFKANRLLHLAYLHVKIILYRPFIHYISEKDKVGNSSIPPSPEEITSIEKAKNCVNVARIVVKLAEDMINRKMLSGSYWFSIYTIFFSVACLVYYVHFAPPKKDNGELDPQYMEIKKDTESGREVLNILKDSSMAARRTYNILNSLFEQLNRRTAKVNLAKAQQPPSGLNNPAATQYQKQGEHRQLQPSNYSGTVKSVDPENIDYSSFGSQFENTNIEDGSSNTKIDQKVNGVNYIDGVFTGINLNMPNLSETSNTQGIDNPAFQSINNSNLNNNFVQTKYIPGMMDQLDMKIFGRFLPPYMLNSNKVEQGQNERNLSGQPSSSNTPDGSQPVTVLDGLYPLQNDNNNNHDPGNSKSVVNNSNSVENLLQNFTMVPSGLSSTVQNPEAAQKFNNHMSNISNMNDPRRASVATSDGSNDMDHHSQGPINKDLKPLSNYEFDDLFFNDWTTAPDTINFDS

SEQ ID NO: 13 - DNA, 534 bp, Saccharomyces cerevisiae
  source 1..534: mol_type = other DNA
atgagcagcattccagctggcactgatcctgggtcctgcggtgctaatttcaagaatgaccgcaagcgcagagataagatcaacgaccgtattcaagaactattgagtatcattcccaaagacttctttagagattattacggcaattctggtagcaatgacacgttaagtgaatccactcccggtgcgcttgggttgtccagcaaggccaaaggtacagggaccaaggacggaaagcccaacaagggccaaattctcacacaggcggtagagtacatatcacatctacaaaatcaagtggacacacagaacagagaggaggtggaactgatggtgaaggccactcagttggccaagcagacaggcaccattgtcaacgatataaacttagagaacaccagcgctgaagtcgccctgtccaggattggcgtgggaccgctggccgcaacaaatgatgactcagtaagaccgccagcaaagaggttgagctccttcgagtacggagggtatggtgagtacggtaatggtagctaa

SEQ ID NO: 14 - protein, 177 aa, Saccharomyces cerevisiae
MSSIPAGTDPGSCGANFKNDRKRRDKINDRIQELLSIIPKDFFRDYYGNSGSNDTLSESTPGALGLSSKAKGTGTKDGKPNKGQILTQAVEYISHLQNQVDTQNREEVELMVKATQLAKQTGTIVNDINLENTSAEVALSRIGVGPLAATNDDSVRPPAKRLSSFEYGGYGEYGNGS

SEQ ID NO: 15 - DNA, 1767 bp, Saccharomyces cerevisiae
  source 1..1767: mol_type = other DNA
atgtcaacacttagcgatagtgataccgagactgaggtcgtgtcgagaaacttgtgtggaatcgtcgacataggttctaatggtattcgttttagtatatcttccaaggctgcacatcatgcaagaattatgccttgtgtttttaaagatagggttggtctttctctatacgaagttcaatataatacacatacgaacgcaaaatgccctattcccagagatattataaaagaggtttgttctgccatgaagagattcaaattaatttgcgatgattttggtgtacctgaaactagtgtcagagtaattgcaacagaagccacgcgagatgctattaacgcggatgaatttgttaatgctgtttacggtagcactggctggaaagtagaaatattaggccaggaagatgaaactagggtcggcatatatggtgttgtttcctcatttaatacagtaagaggtctatatctagatgtggcaggtggtagtactcagttatcatgggtaataagctcgcacggagaagtcaagcaatccagcaaacctgtatctttgccatatggagctggaactcttttgagaagaatgagaacagatgataatagggcacttttttatgagattaaagaagcgtacaaagatgcgattgaaaaaattggtatacctcaagaaatgattgatgacgccaagaaagaaggtggatttgacctttggacccgtgggggtggtttaagaggtatgggacatctgcttctttaccagtcggaaggttatcccatccaaacaataattaacggatatgcttgcacttatgaagaattctcgtctatgtcagattatctattcctaaaacaaaaaataccaggttcttcaaaagagcataaaatatttaaggtttctgatagaagggctttacaacttcctgccgttggtttgttcatgagtgctgtttttgaagcgattccccagatcaaagctgtacattttagtgagggtggtgttcgagagggttcactttattctcttcttccaaaagaaattcgtgcacaagatccattgctaattgcgtcccgtccttatgctccattacttactgaaaaatatctatatctattgagaacatcaatcccacaagaagatataccagaaatagtaaacgaaaggattgctcctgctttatgtaacttagcatttgttcatgcctcttatccaaaggagttacaaccaacagctgcattacatgttgctacaagagggataatagccggctgtcatggattatctcacagagctagagcgctgataggaattgctctatgtagtagatggggcggcaacattccggaatctgaagaaaaatactcccaagaattagaacaagtagttctacgcgaaggtgataaagctgaagcattgagaattgtatggtggacgaagtatattggtacgattatgtatgtgatttgcggtgttcatccaggtggtaatatcagagataacgtatttgatttccatgtttctaagcgtagtgaggtggagaccagtttaaaagaattaatcattgatgatgcaaacactacaaaggtaaaagaagaatccacgcgtaaaaatcgcgggtatgaagtggttgtgagaattagtaaggacgatcttaaaacaagtgcttccgttcgttccagaattatcacgctacaaaagaaagtacgcaagctatctagaggaagtgtagagagggttaaaattggcgtgcaattttatgaagaataa

SEQ ID NO: 16 - protein, 588 aa, Saccharomyces cerevisiae
MSTLSDSDTETEVVSRNLCGIVDIGSNGIRFSISSKAAHHARIMPCVFKDRVGLSLYEVQYNTHTNAKCPIPRDIIKEVCSAMKRFKLICDDFGVPETSVRVIATEATRDAINADEFVNAVYGSTGWKVEILGQEDETRVGIYGVVSSFNTVRGLYLDVAGGSTQLSWVISSHGEVKQSSKPVSLPYGAGTLLRRMRTDDNRALFYEIKEAYKDAIEKIGIPQEMIDDAKKEGGFDLWTRGGGLRGMGHLLLYQSEGYPIQTIINGYACTYEEFSSMSDYLFLKQKIPGSSKEHKIFKVSDRRALQLPAVGLFMSAVFEAIPQIKAVHFSEGGVREGSLYSLLPKEIRAQDPLLIASRPYAPLLTEKYLYLLRTSIPQEDIPEIVNERIAPALCNLAFVHASYPKELQPTAALHVATRGIIAGCHGLSHRARALIGIALCSRWGGNIPESEEKYSQELEQVVLREGDKAEALRIVWWTKYIGTIMYVICGVHPGGNIRDNVFDFHVSKRSEVETSLKELIIDDANTTKVKEESTRKNRGYEVVVRISKDDLKTSASVRSRIITLQKKVRKLSRGSVERVKIGVQFYEE

SEQ ID NO: 17 - protein, 161 aa, Vigna radiata
MTTTLERGFTEEQEALVVKSWNVMKKNSGELGLKFFLKIFEIAPSAQKLFSFLRDSTVPLEQNPKLKPHAVSVFVMTCDSAVQLRKAGKVTVRESNLKKLGATHFRTGVANEHFEVTKFALLETIKEAVPEMWSPAMKNAWGEAYDQLVDAIKYEMKPPSS

SEQ ID NO: 18 - protein, 133 aa, Methylacidiphilum infernorum
MIDQKEKELIKESWKRIEPNKNEIGLLFYANLFKEEPTVSVLFQNPISSQSRKLMQVLGILVQGIDNLEGLIPTLQDLGRRHKQYGVVDSHYPLVGDCLLKSIQEYLGQGFTEEAKAAWTKVYGIAAQVMTAE

SEQ ID NO: 19 - protein, 139 aa, Aquifex aeolicus
MLSEETIRVIKSTVPLLKEHGTEITARMYELLFSKYPKTKELFAGASEEQPKKLANAIIAYATYIDRLEELDNAISTIARSHVRRNVKPEHYPLVKECLLQAIEEVLNPGEEVLKAWEEAYDFLAKTLITLEKKLYSQP

SEQ ID NO: 20 - protein, 145 aa, Glycine max
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 21 - protein, 162 aa, Hordeum vulgare
MSAAEGAVVFSEEKEALVLKSWAIMKKDSANLGLRFFLKIFEIAPSARQMFPFLRDSDVPLETNPKLKTHAVSVFVMTCEAAAQLRKAGKITVRETTLKRLGGTHLKYGVADGHFEVTRFALLETIKEALPADMWGPEMRNAWGEAYDQLVAAIKQEMKPAE

SEQ ID NO: 22 - protein, 1153 aa, Magnaporthe oryzae
MDGAVRLDWTGLDLTGHEIHDGVPIASRVQVMVSFPLFKDQHIIMSSKESPSRKSSTIGQSTRNGSCQADTQKGQLPPVGEKPKPVKENPMKKLKEMSQRPLPTQHGDGTYPTEKKLTGIGEDLKHIRGYDVKTLLAMVKSKLKGEKLKDDKTMLMERVMQLVARLPTESKKRAELTDSLINELWESLDHPPLNYLGPEHSYRTPDGSYNHPFNPQLGAAGSRYARSVIPTVTPPGALPDPGLIFDSIMGRTPNSYRKHPNNVSSILWYWATIIIHDIFWTDPRDINTNKSSSYLDLAPLYGNSQEMQDSIRTFKDGRMKPDCYADKRLAGMPPGVSVLLIMFNRFHNHVAENLALINEGGRFNKPSDLLEGEAREAAWKKYDNDLFQVARLVTSGLYINITLVDYVRNIVNLNRVDTTWTLDPRQDAGAHVGTADGAERGTGNAVSAEFNLCYRWHSCISEKDSKFVEAQFQNIFGKPASEVRPDEMWKGFAKMEQNTPADPGQRTFGGFKRGPDGKFDDDDLVRCISEAVEDVAGAFGARNVPQAMKVVETMGIIQGRKWNVAGLNEFRKHFHLKPYSTFEDINSDPGVAEALRRLYDHPDNVELYPGLVAEEDKQPMVPGVGIAPTYTISRVVLSDAVCLVRGDRFYTTDFTPRNLTNWGYKEVDYDLSVNHGCVFYKLFIRAFPNHFKQNSVYAHYPMVVPSENKRILEALGRADLFDFEAPKYIPPRVNITSYGGAEYILETQEKYKVTWHEGLGFLMGEGGLKFMLSGDDPLHAQQRKCMAAQLYKDGWTEAVKAFYAGMMEELLVSKSYFLGNNKHRHVDIIRDVGNMVHVHFASQVFGLPLKTAKNPTGVFTEQEMYGILAAIFTTIFFDLDPSKSFPLRTKTREVCQKLAKLVEANVKLINKIPWSRGMFVGKPAKDEPLSIYGKTMIKGLKAHGLSDYDIAWSHVVPTSGAMVPNQAQVFAQAVDYYLSPAGMHYIPEIHMVALQPSTPETDALLLGYAMEGIRLAGTFGSYREAAVDDVVKEDNGRQVPVKAGDRVFVSFVDAARDPKHFPDPEVVNPRRPAKKYIHYGVGPHACLGRDASQIAITEMFRCLFRRRNVRRVPGPQGELKKVPRPGGFYVYMREDWGGLFPFPVTMRVMWDDE

SEQ ID NO: 23 - protein, 530 aa, Fusarium oxysporum
MKGSATLAFALVQFSAASQLVWPSKWDEVEDLLYMQGGFNKRGFADALRTCEFGSNVPGTQNTAEWLRTAFHDAITHDAKAGTGGLDASIYWESSRPENPGKAFNNTFGFFSGFHNPRATASDLTALGTVLAVGACNGPRIPFRAGRIDAYKAGPAGVPEPSTNLKDTFAAFTKAGFTKEEMTAMVACGHAIGGVHSVDFPEIVGIKADPNNDTNVPFQKDVSSFHNGIVTEYLAGTSKNPLVASKNATFHSDKRIFDNDKATMKKLSTKAGFNSMCADILTRMIDTVPKSVQLTPVLEAYDVRPYITELSLNNKNKIHFTGSVRVRITNNIRDNNDLAINLIYVGRDGKKVTVPTQQVTFQGGTSFGAGEVFANFEFDTTMDAKNGITKFFIQEVKPSTKATVTHDNQKTGGYKVDDTVLYQLQQSCAVLEKLPNAPLVVTAMVRDARAKDALTLRVAHKKPVKGSIVPRFQTAITNFKATGKKSSGYTGFQAKTMFEEQSTYFDIVLGGSPASGVQFLTSQAMPSQCS

SEQ ID NO: 24 - protein, 358 aa, Fusarium graminearum
MASATRQFARAATRATRNGFAIAPRQVIRQQGRRYYSSEPAQKSSSAWIWLTGAAVAGGAGYYFYGNSASSATAKVFNPSKEDYQKVYNEIAARLEEKDDYDDGSYGPVLVRLAWHASGTYDKETGTGGSNGATMRFAPESDHGANAGLAAARDFLQPVKEKFPWITYSDLWILAGVCAIQEMLGPAIPYRPGRSDRDVSGCTPDGRLPDASKRQDHLRGIFGRMGFNDQEIVALSGAHALGRCHTDRSGYSGPWTFSPTVLTNDYFRLLVEEKWQWKKWNGPAQYEDKSTKSLMMLPSDIALIEDKKFKPWVEKYAKDNDAFFKDFSNVVLRLFELGVPFAQGTENQRWTFKPTHQE

SEQ ID NO: 25 - protein, 122 aa, Chlamydomonas eugametos
MSLFAKLGGREAVEAAVDKFYNKIVADPTVSTYFSNTDMKVQRSKQFAFLAYALGGASEWKGKDMRTAHKDLVPHLSDVHFQAVARHLSDTLTELGVPPEDITDAMAVVASTRTEVLNMPQQ

SEQ ID NO: 26 - protein, 121 aa, Tetrahymena pyriformis
MNKPQTIYEKLGGENAMKAAVPLFYKKVLADERVKHFFKNTDMDHQTKQQTDFLTMLLGGPNHYKGKNMTEAHKGMNLQNLHFDAIIENLAATLKELGVTDAVINEAAKVIEHTRKDMLGK

SEQ ID NO: 27 - protein, 117 aa, Paramecium caudatum
MSLFEQLGGQAAVQAVTAQFYANIQADATVATFFNGIDMPNQTNKTAAFLCAALGGPNAWTGRNLKEVHANMGVSNAQFTTVIGHLRSALTGAGVAAALVEQTVAVAETVRGDVVTV

SEQ ID NO: 28 - protein, 147 aa, Aspergillus niger
MPLTPEQIKIIKATVPVLQEYGTKITTAFYMNMSTVHPELNAVFNTANQVKGHQARALAGALFAYASHIDDLGALGPAVELICNKHASLYIQADEYKIVGKYLLEAMKEVLGDACTDDILDAWGAAYWALADIMINREAALYKQSQG

SEQ ID NO: 29 - protein, 165 aa, Zea mays
MALAEADDGAVVFGEEQEALVLKSWAVMKKDAANLGLRFFLKVFEIAPSAEQMFSFLRDSDVPLEKNPKLKTHAMSVFVMTCEAAAQLRKAGKVTVRETTLKRLGATHLRYGVADGHFEVTGFALLETIKEALPADMWSLEMKKAWAEAYSQLVAAIKREMKPDA

SEQ ID NO: 30 - protein, 169 aa, Oryza sativa
MALVEGNNGVSGGAVSFSEEQEALVLKSWAIMKKDSANIGLRFFLKIFEVAPSASQMFSFLRNSDVPLEKNPKLKTHAMSVFVMTCEAAAQLRKAGKVTVRDTTLKRLGATHFKYGVGDAHFEVTRFALLETIKEAVPVDMWSPAMKSAWSEAYNQLVAAIKQEMKPAE

SEQ ID NO: 31 - protein, 160 aa, Arabidopsis thaliana
MESEGKIVFTEEQEALVVKSWSVMKKNSAELGLKLFIKIFEIAPTTKKMFSFLRDSPIPAEQNPKLKPHAMSVFVMCCESAVQLRKTGKVTVRETTLKRLGASHSKYGVVDEHFEVAKYALLETIKEAVPEMWSPEMKVAWGQAYDHLVAAIKAEMNLSN

SEQ ID NO: 32 - protein, 147 aa, Pisum sativum
MGFTDKQEALVNSSWESFKQNLSGNSILFYTIILEKAPAAKGLFSFLKDTAGVEDSPKLQAHAEQVFGLVRDSAAQLRTKGEVVLGNATLGAIHVQRGVTDPHFVVVKEALLQTIKKASGNNWSEELNTAWEVAYDGLATAIKKAMT

SEQ ID NO: 33 - protein, 145 aa, Vigna unguiculata
MVAFSDKQEALVNGAYEAFKANIPKYSVVFYTTILEKAPAAKNLFSFLANGVDATNPKLTGHAEKLFGLVRDSAAQLRASGGVVADAALGAVHSQKAVNDAQFVVVKEALVKTLKEAVGDKWSDELGTAVELAYDELAAAIKKAY

SEQ ID NO: 34 - protein, 154 aa, Bos taurus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 35 - protein, 154 aa, Sus scrofa
MGLSDGEWQLVLNVWGKVEADVAGHGQEVLIRLFKGHPETLEKFDKFKHLKSEDEMKASEDLKKHGNTVLTALGGILKKKGHHEAELTPLAQSHATKHKIPVKYLEFISEAIIQVLQSKHPGDFGADAQGAMSKALELFRNDMAAKYKELGFQG

SEQ ID NO: 36 - protein, 154 aa, Equus caballus
MGLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

SEQ ID NO: 37 - protein, 152 aa, Nicotiana benthamiana
MSSFTEEQEALVLKSWDSMKKNAGEWGLKLFLKIFEIAPSAKKLFSFLKDSNVPLEQNAKLKPHAKSVFVMTCEAAVQLRKAGKVVVRDSTLKKLGAAHFKYGVADEHFEVTKFALLETIKEAVPDMWSVDMKNAWGEAFDQLVNGIKTEMK

SEQ ID NO: 38 - protein, 132 aa, Bacillus subtilis
MGQSFNAPYEAIGEELLSQLVDTFYERVASHPLLKPIFPSDLTETARKQKQFLTQYLGGPPLYTEEHGHPMLRARHLPFPITNERADAWLSCMKDAMDHVGLEGEIREFLFGRLELTARHMVNQTEAEDRSS

SEQ ID NO: 39 - protein, 131 aa, Corynebacterium glutamicum
MTTSENFYDSVGGEETFSLIVHRFYEQVPNDDILGPMYPPDDFEGAEQRLKMFLSQYWGGPKDYQEQRGHPRLRMRHVNYPIGVTAAERWLQLMSNALDGVDLTAEQREAIWEHMVRAADMLINSNPDPHA

SEQ ID NO: 40 - protein, 124 aa, Synechocystis sp.
MSTLYEKLGGTTAVDLAVDKFYERVLQDDRIKHFFADVDMAKQRAHQKAFLTYAFGGTDKYDGRYMREAHKELVENHGLNGEHFDAVAEDLLATLKEMGVPEDLIAEVAAVAGAPAHKRDVLNQ

SEQ ID NO: 41 - protein, 183 aa, Synechococcus sp.
MDVALLEKSFEQISPRAIEFSASFYQNLFHHHPELKPLFAETSQTIQEKKLIFSLAAIIENLRNPDILQPALKSLGARHAEVGTIKSHYPLVGQALIETFAEYLAADWTEQLATAWVEAYDVIASTMIEGADNPAAYLEPELTFYEWLDLYGEESPKVRNAIATLTHFHYGEDPQDVQRDSRG

SEQ ID NO: 42 - protein, 118 aa, Nostoc commune
MSTLYDNIGGQPAIEQVVDELHKRIATDSLLAPVFAGTDMVKQRNHLVAFLAQIFEGPKQYGGRPMDKTHAGLNLQQPHFDAIAKHLGERMAVRGVSAENTKAALDRVTNMKGAILNK

SEQ ID NO: 43 - protein, 136 aa, Bacillus megaterium
MREKIHSPYELLGGEHTISKLVDAFYTRVGQHPELAPIFPDNLTETARKQKQFLTQYLGGPSLYTEEHGHPMLRARHLPFEITPSRAKAWLTCMHEAMDEINLEGPERDELYHRLILTAQHMINSPEQTDEKGFSH

SEQ ID NO: 44 - DNA, 934 bp, synthetic construct
  source 1..934: mol_type = other DNA
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg